(19) United States
(12) Patent Application Publication
(10) Pub. No.: US 2010/0188491 A1
(43) Pub. Date: Jul. 29, 2010

(54) SOLID-STATE IMAGING DEVICE, ENDOSCOPE APPARATUS, AND DRIVE METHOD OF SOLID-STATE IMAGING DEVICE

(75) Inventor: Makoto SHIZUKUISHI, Kanagawa (JP)

Correspondence Address: Studebaker & Brackett PC, One Fountain Square, 11911 Freedom Drive, Suite 750, Reston, VA, 20190, US

(73) Assignee: FUJIFILM CORPORATION, Tokyo (JP)

(21) Appl. No.: 12/693,856

(22) Filed: Jan. 26, 2010

(30) Foreign Application Priority Data
Jan. 26, 2009 (JP) 2009-014230
May 27, 2009 (JP) 2009-127950

(51) Int. Cl. H04N 7/18; H04N 5/335

(52) U.S. Cl. 348/65; 348/311; 348/E07.085; 348/E05.091

(57) ABSTRACT

A solid-state imaging device includes plural pixel portions and a signal reading section. Each of the plural pixel portions includes a photoelectric conversion portion, and plural charge storage portions a selected one of which can store charge generated in the photoelectric conversion portion. The signal reading section independently reads out signals corresponding to amounts of charges stored in the plural respective charge storage portions.

### CROSS-REFERENCE TO RELATED APPLICATIONS

This application is based on and claims priority under 35 USC 119 from Japanese Patent Application Nos. 2009-014230 filed on Jan. 26, 2009 and 2009-127950 filed on May 27, 2009; the entire of which are incorporated herein by reference.

### BACKGROUND

1. Technical Field

The present invention relates to a solid-state imaging device having plural pixel portions, an endoscope apparatus incorporating it, and its drive method.

2. Related Art

In many cases, living body information that is necessary to detect early-stage cancer is hard to see by ordinary naked-eye observation. To increase the diagnosis accuracy, it is very effective to visualize plural pieces of living body information. In view of this, in shooting of the inside of a body using an electronic endoscope, in addition to ordinary shooting which uses a light source of white light, special shooting which uses a light source of special light (e.g. infrared light) may be performed to take a more clear image of an object (obtain more clear living body information) which is difficult to discriminate by ordinary shooting.

Patent document 1 (JP-A-2001-087221) discloses the following technique. An infrared cutting filter which transmits visible light and cuts infrared light and an infrared transmission filter which transmits infrared light and cuts visible light can be inserted before a solid-state imaging device in a switched manner. Ordinary shooting is performed by applying visible light to an object in a state that the infrared cutting filter is inserted before the solid-state imaging device, and an ordinary visible image is obtained from a signal thus produced. Then, special shooting is performed by applying infrared light to the object in a state that the infrared transmission filter is inserted before the solid-state imaging device, and an infrared image is obtained from a signal thus produced.

In this method, since signal reading needs to be performed between ordinary shooting and special shooting, a time difference occurs between the ordinary shooting and the special shooting. As a result, if, for example, an object moves, the two kinds of shooting operations cannot be performed under the same conditions and correct comparison cannot be performed for the object.

### SUMMARY

an illustrative aspect of the invention is to provide a solid-state imaging device, an endoscope apparatus, and a drive method of a solid-state imaging device which make it possible to perform ordinary shooting and special shooting approximately at the same time.

According to an aspect of the invention, a solid-state imaging device includes plural pixel portions and a signal reading section. Each of the plural pixel portions includes: a photoelectric conversion portion; and plural charge storage portions a selected one of which can store charge generated in the photoelectric conversion portion. The signal reading section independently reads out signals corresponding to amounts of charges stored in the plural respective charge storage portions.

According to another aspect of the invention, an endoscope apparatus includes: said solid-state imaging device; a light source capable of emitting plural kinds of light; and a drive unit that performs drives for causing emissions of the plural kinds of light at different time points in response to a shooting trigger, causing charges generated in the photoelectric conversion portion by incident light beams coming from an object as a result of the emissions to be stored in the different charge storage portions corresponding to the plural respective kinds of light in synchronism with the emissions of the plural kinds of light, and causing the signal reading section to start reading out signals corresponding to amounts of the charges stored in the plural charge storage portions, respectively.

According to another aspect of the invention, it is a drive method of a solid-state imaging device having plural pixel portions. Each of the pixel portions includes a photoelectric conversion portion and plural charge storage portions capable of storing, at different time points, charges generated in the photoelectric conversion portion. The drive method includes: causing emissions of plural kinds of light at different time points in response to a shooting trigger, and causing charges generated in the photoelectric conversion portion by incident light beams coming from an object as a result of the emissions to be stored in the different charge storage portions corresponding to the plural respective kinds of light in synchronism with the emissions of the plural kinds of light; and starting to read out signals corresponding to amounts of the charges stored in the plural charge storage portions, respectively.

With the above-mentioned configurations of the solid-state imaging device, the endoscope apparatus, and the drive method of the solid-state imaging device, it is possible to perform ordinary shooting and special shooting approximately at the same time.

### BRIEF DESCRIPTION OF THE DRAWINGS

FIG. 1 shows a general configuration of an endoscope apparatus according to an embodiment of the present invention.

FIGS. 2A and 2B show a general configuration of a solid-state imaging device shown in FIG. 1.

FIG. 3 is an equivalent circuit diagram showing the internal structure of each pixel portion shown in FIG. 2A.

FIG. 4 is a schematic plan view showing an example layout of two pixel portions each of which corresponds to the equivalent circuit diagram of FIG. 3.

FIG. 5 is a graph showing relationships between spectral characteristics of color filters and emission lines of special light beams.

FIG. 6 is a timing chart showing an operation of the endoscope apparatus of FIG. 1.

FIG. 7 is a schematic diagram showing the operation of the endoscope apparatus of FIG. 1.

FIGS. 8A and 8B show a general configuration of another solid-state imaging device according to another embodiment of the invention.

FIG. 9 is an equivalent circuit diagram of each pixel portion of the solid-state imaging device of FIG. 8A.

FIG. 10 is a schematic plan view showing an example layout of each pixel portion of the solid-state imaging device of FIG. 8A.

FIG. 11 is a schematic sectional view of the pixel portion taken along line A-A′ in FIG. 10.

FIG. 12 is a schematic sectional view of the pixel portion taken along line B-B′ in FIG. 10.

FIG. 13 shows a modification of the solid-state imaging device of FIGS. 8A and 8B.

FIG. 14 shows a first modification of the endoscope apparatus of FIG. 1 and is an equivalent circuit diagram showing a modified structure of each pixel portion.

FIG. 15 is a timing chart showing an operation of a second modification of the imaging apparatus of FIG. 1.

FIG. 16 is a schematic diagram showing the operation of the second modification of the endoscope apparatus of FIG. 1.

### DETAILED DESCRIPTION

Exemplary embodiments of the present invention will be hereinafter described with reference to the drawings.

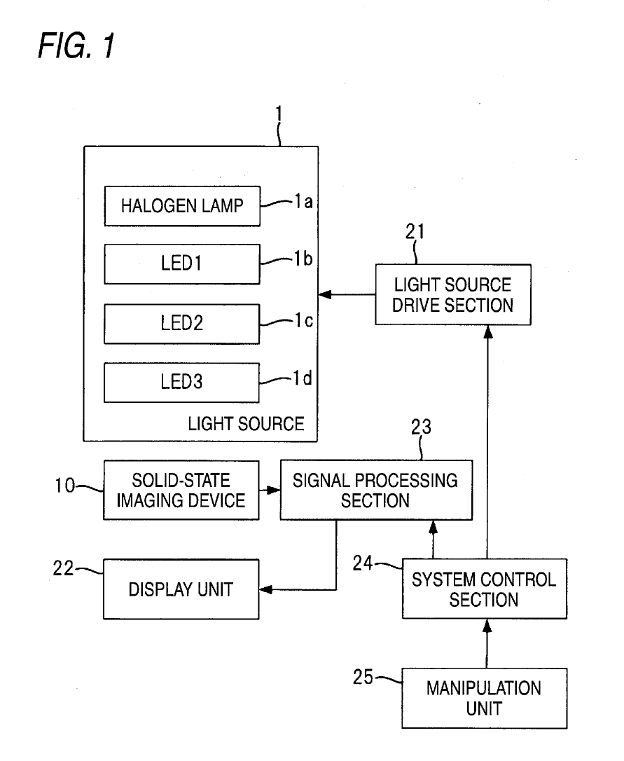

FIG. 1 shows a general configuration of an endoscope apparatus according to an embodiment of the invention. The endoscope apparatus of FIG. 1 is equipped with a light source 1, a solid-state imaging device 10, a light source drive section 21, a signal processing section 23, a system control section 24, a display unit 22, and a manipulation unit 25.

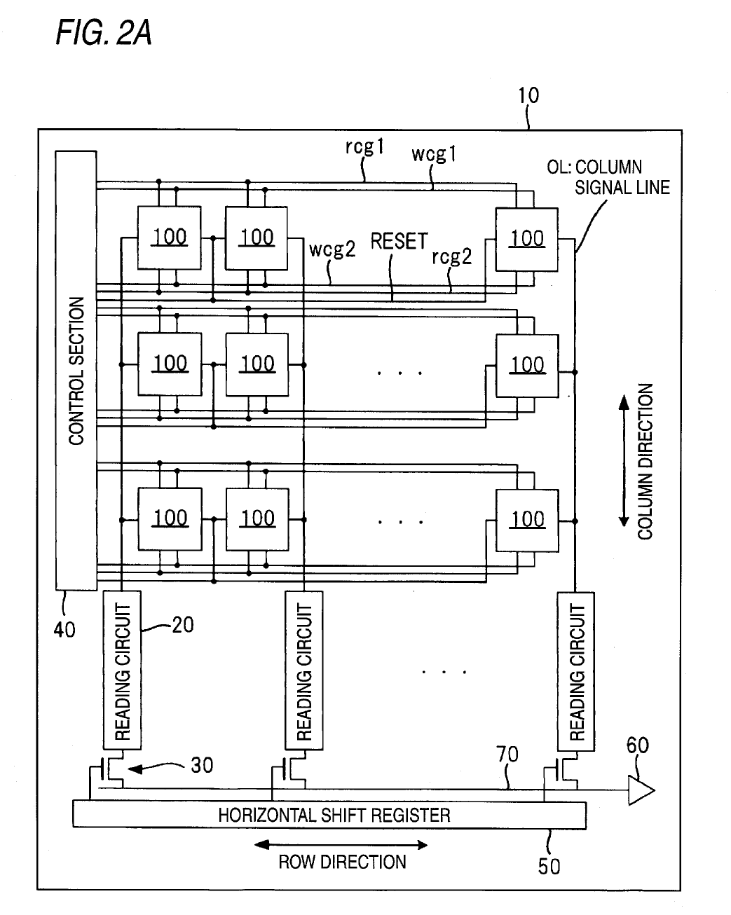

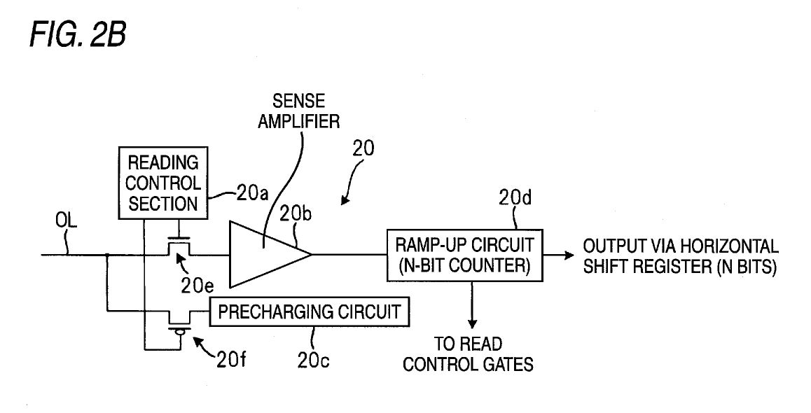

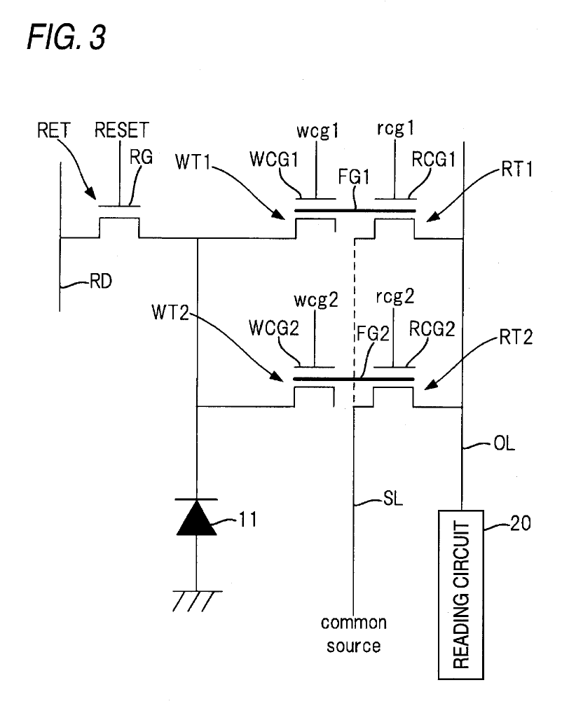

FIGS. 2A and 2B show a general configuration of the solid-state imaging device 10 shown in FIG. 1. FIG. 3 is an equivalent circuit diagram showing the internal structure of each pixel portion 100 shown in FIG. 2A.

The solid-state imaging device 10 is provided with plural pixel portions 100 which are arranged in the same plane in a row direction and a column direction that is perpendicular to the row direction so as to form an array (in this example, a square lattice).

Each pixel portion 100 is provided with an n-type impurity layer 11 which is formed in a p-well layer which is formed in an n-type silicon substrate. The n-type silicon substrate and the p-well layer constitute a semiconductor substrate. The n-type impurity layer 11 forms a pn junction with the p-well layer and thereby forms a photodiode (PD) which functions as a photoelectric conversion portion. In the following description, the n-type impurity layer 11 will be referred to as “photoelectric conversion portion 11.”

Two charge storage portions (examples of “plural storage portions”), a selected one of which can store charge generated by the photoelectric conversion portion 11, are formed in each pixel portion 100. In the following, the two charge storage portions will be referred to as a first charge storage portion and a second charge storage portion.

The first charge storage portion is provided with a write transistor WT1 and a read transistor RT1.

The write transistor WT1 is a MOS transistor having a two-terminal structure in which a floating gate FG1 which electrically floats functions as a charge storage region and the photoelectric conversion portion 11 serves as the source and the drain. The operation of the write transistor WT1 is controlled by a write control gate WCG1, which is connected to a control section 40 by a write control line wcg1. When a write pulse is applied to the write control gate WCG1 of the write transistor WT1, charge generated by the photoelectric conversion portion 11 is injected into the floating gate FG1 through F-N tunneling injection, direct tunneling injection, hot electron injection, or the like and stored there. Alternatively, the write transistor WT1 may have a three-terminal structure in which the photoelectric conversion portion 11 serves as the source and the drain is provided separately.

The read transistor RT1 is a MOS transistor which shares the floating gate FG1 with the write transistor WT1 and shares the source with a read transistor RT2 (described later) and in which a column signal line OL is connected to the drain. The operation of the read transistor RT1 is controlled by a read control gate RCG1, which is connected to the control section 40 by a read control line rcg1. The threshold voltage of the read transistor RT1 is varied according to the amount of charge stored in the floating gate FG1. Therefore, a change in the threshold voltage (i.e., a change with respect to a threshold voltage of a case that no charge is stored in the floating gate FG1) can be read out as an imaging signal.

The structure of each pixel portion 100 is not limited to the one in which the floating gate FG1 is shared by the write transistor WT1 and the read transistor RT1. Another structure is possible in which two separate floating gates FG1 are formed for the write transistor WT1 and the read transistor RT1 and electrically connected to each other by an interconnection.

The second charge storage portion is provided with a write transistor WT2 and a read transistor RT2.

The write transistor WT2 is a MOS transistor having a two-terminal structure in which a floating gate FG1 which electrically floats functions as a charge storage region and the photoelectric conversion portion 11 serves as the source and the drain. The operation of the write transistor WT2 is controlled by a write control gate WCG2, which is connected to the control section 40 by a write control line wcg2. When a write pulse is applied to the write control gate WCG2 of the write transistor WT2, charge generated by the photoelectric conversion portion 11 is injected into the floating gate FG2 through FN tunneling injection, direct tunneling injection, hot electron injection, or the like and stored there. Alternatively, the write transistor WT2 may also have a three-terminal structure in which the photoelectric conversion portion 11 serves as the source and the drain is provided separately. In this case, the write transistors WT1 and WT2 share the drain.

The read transistor RT2 is a MOS transistor which shares the floating gate FG2 with the write transistor WT2 and shares the source with the read transistor RT1 and in which the column signal line OL is connected to the drain. The operation of the read transistor RT2 is controlled by a read control gate RCG2, which is connected to the control section 40 by a read control line rcg2. The threshold voltage of the read transistor RT2 is varied according to the amount of charge stored in the floating gate FG2. Therefore, a change in the threshold voltage (i.e., a change with respect to a threshold voltage of a case that no charge is stored in the floating gate FG2) can be read out as an imaging signal.

The structure of each pixel portion 100 is not limited to the one in which the floating gate FG2 is shared by the write transistor WT2 and the read transistor RT2. Another structure is possible in which two separate floating gates FG2 are formed for the write transistor WT2 and the read transistor RT2 and electrically connected to each other by an interconnection.

The common source of the read transistors RT1 and RT2 is given a prescribed potential via a source line SL.

Each pixel portion 100 is also provided with a reset transistor RET for ejecting the charge stored in the photoelectric conversion portion 11. A reset gate RG of the reset transistor RET is connected to the control section 40 by a reset line RESET. When a reset pulse is applied to the reset gate RG from the control section 40 via the reset line RESET, the reset transistor RET is turned on and the charge stored in the photoelectric conversion portion 11 is ejected to the drain of the reset transistor RET. The drain of the reset transistor RET is supplied with a power source voltage Vcc by a reset drain line RD.

The solid-state imaging device 10 is equipped with the control section 40 for drive-controlling the individual pixel portions 100, reading circuits 20 for detecting threshold voltages of the read transistors RT1 and RT2, a horizontal shift register 50 and horizontal selection transistors 30 for performing a control for sequentially reading out, to a signal line 70, as imaging signals, threshold voltages of one line detected by the respective reading circuits 20, and an output amplifier 60 which is connected to the signal line 70.

The reading circuits 20 are provided for the respective sets of pixel portions 100 that are arranged in the column direction. Each reading circuit 20 is connected by the column signal line OL to the drains of the read transistors RT1 and RT2 of the pixel portions 100 of the corresponding column. The reading circuits 20 are also connected to the control section 40.

As shown in FIG. 2B, each reading circuit 20 is provided with a reading control section 20a, a sense amplifier 20b, a precharging circuit 20c, a ramp-up circuit 20d, and transistors 20e and 20f.

In reading out a signal from the first charge storage portion (or second charge storage portion) of a pixel portion 100, the reading control section 20a turns on the transistor 20f and thereby causes the precharging circuit 20c to supply a drain voltage to the drain of the read transistor RT1 (or read transistor RT2) of the pixel portion 100 via the column signal line OL (precharging). Then, the reading control section 20a turns on the transistor 20e and thereby establishes electrical continuity between the drain of the read transistor RT1 (or read transistor RT2) of the pixel portion 100 and the sense amplifier 20b.

Monitoring the drain voltage of the read transistor RT1 (or read transistor RT2) of the pixel portion 100, the sense amplifier 20b detects a change in the drain voltage and informs the ramp-up circuit 20d of that fact. For example, the sense amplifier 20b detects a drop from the drain voltage value produced by the precharging by the precharging circuit 20c and inverts its output.

Incorporating an N-bit counter (e.g., N=8 to 12), the ramp-up circuit 20d supplies a gradually increasing or decreasing ramp voltage to the read control gate RCG1 (or read control gate RCG2) of the read transistor RT1 (or read transistor RT2) of the pixel portion 100 via the control section 40 and outputs a count (a combination of 1s and 0s numbering N together) that corresponds to the ramp voltage value.

If the voltage of the read control gate RCG1 (or read control gate RCG2) exceeds the threshold voltage of the read transistor RT1 (or read transistor RT2), the read transistor RT1 (or read transistor RT2) is turned on and the potential of the column signal line OL drops which has been precharged. The sense amplifier 20b detects this event and outputs an inverted signal. The ramp-up circuit 20d latches a count corresponding to a ramp voltage value being generated at the time point of reception of the inverted signal. In this manner, a change in the threshold voltage can be read out as a digital value (a combination of 1s and 0s; imaging signal).

When one horizontal selection transistor 30 is selected by the horizontal shift register 50, a count that is latched by the ramp-up circuit 20d connected to the selected horizontal selection transistor 30 is output to the signal line 70 and output from the output amplifier 60 as an imaging signal.

The method for reading out a change in the threshold voltage of the read transistor RT1 (or read transistor RT2) by the reading circuit 20 is not limited to the above method. For example, a drain current of the read transistor RT1 (or read transistor RT2) that occurs when a prescribed voltage is applied between the read control gate RCG1 (or read control gate RCG2) and the drain of the read transistor RT1 (or read transistor RT2) may be read out.

The control section 40 performs a drive for injecting charge generated by the photoelectric conversion portion 11 into the floating gate FG1 or FG2 and storing the charge there by controlling the write transistors WT1 and WT2 independently. Example methods for injecting charge into the floating gate FG1 or FG2 are CHE injection which uses channel hot electrons (CHEs), Fowler-Nordheim (F-N) tunneling electron injection which uses tunneling current, and direct tunneling electron injection through a thin tunneling oxide layer (about 1 to 5 nm in thickness).

The control section 40 performs a drive for independently reading out an imaging signal corresponding to the charge stored in the floating gate FG1 or FG2 by controlling the reading circuit 20.

Furthermore, the control section 40 performs a drive for ejecting, to the outside, the charge stored in the floating gate FG1 or FG2 and thereby erasing it. For example, the control section 40 erases the charge stored in the floating gate FG1 by drawing it into the semiconductor substrate by applying a positive voltage to the semiconductor substrate and negative voltages to the write control gate WCG1 and the read control gate RCG1. The control section 40 erases the charge stored in the floating gate FG2 by applying a positive voltage to the semiconductor substrate and negative voltages to the write control gate WCG2 and the read control gate RCG2.

Color filters (not shown) are provided over the photoelectric conversion portions 11 of the pixel portions 100. The color filters include blue color filters which transmit blue light (in general, in a wavelength range of about 380 nm to about 520 nm), green color filters which transmit green light (in general, in a wavelength range of about 450 nm to about 610 nm), and red color filters which transmit red light (in general, in a wavelength range of about 550 nm to about 700 nm). The color filters are in a Bayer arrangement.

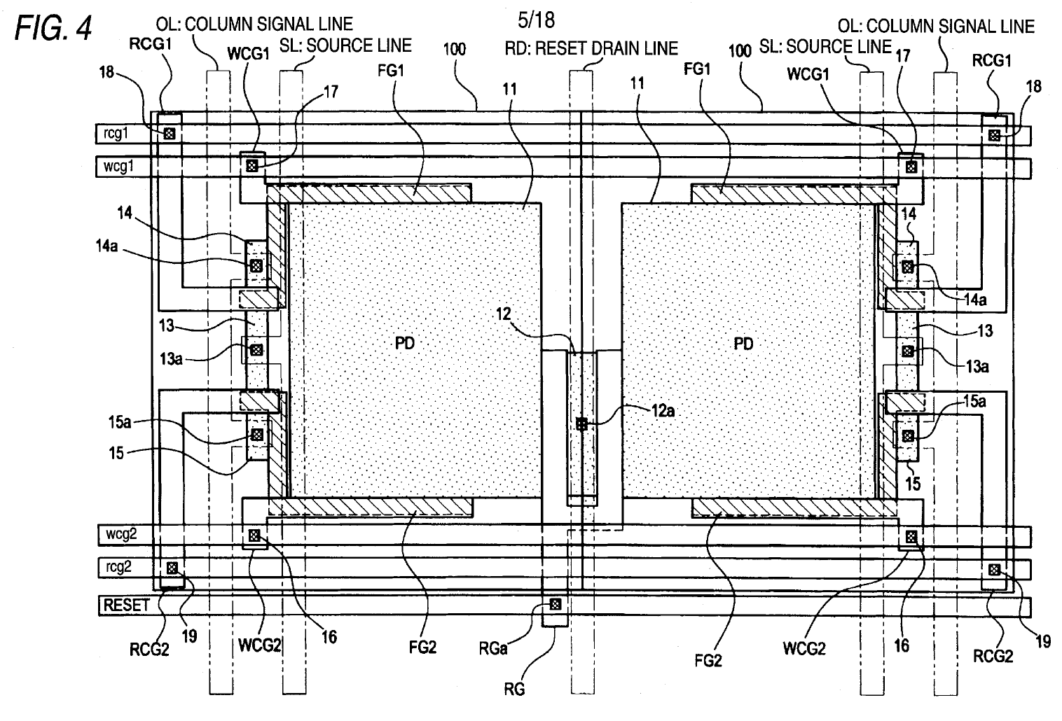

FIG. 4 is a schematic plan view showing an example layout of two pixel portions 100 adjacent to each other in the row direction each of which corresponds to the equivalent circuit diagram of FIG. 3. Each line of the solid-state imaging device 10 is formed in such a manner that plural sets of the two pixel portions 100 shown in FIG. 4 are arranged in the row direction. Since the two pixel portions shown in FIG. 4 are symmetrical with respect to the center line of the drain 12 of the reset transistor RET, only the left-hand pixel portion 100 will be described below.

The photoelectric conversion portion 11 is formed in the semiconductor substrate. And the drain 14 of the read transistor RT1, the common source 13 of the read transistors RT1 and RT2, and the drain 15 of the read transistor RT2 are formed on the left of the photoelectric conversion portion 11 with a small space so as to be arranged in the column direction. The drain 12 of the reset transistor RET is formed on the right of the photoelectric conversion portion 11 with a small space.

An oxide layer (not shown) is formed on the semiconductor substrate and the floating gates FG1 and FG2 are formed on the oxide layer. The floating gate FG1 is formed alongside the top and left sidelines of the photoelectric conversion portion 11 so as to extend to over the portion between the drain 14 and the source 13. The floating gate FG2 is formed alongside the bottom and left sidelines of the photoelectric conversion portion 11 so as to extend to over the portion between the drain 15 and the source 13.

An insulating layer is formed on the floating gates FG1 and FG2, and the write control gates WCG1 and WCG2 and the read control gates RCG1 and RCG2, and the reset gate RG are formed on the insulating layer.

The write control gate WCG1 is formed so as to overlap with the floating gate FG1. The read control gate RCG1 is formed so as to overlap with the portion of the floating gate FG1 that is located over the portion between the drain 14 and the source 13.

The write control gate WCG2 is formed so as to overlap with the floating gate FG2. The read control gate RCG2 is formed so as to overlap with the portion of the floating gate FG2 that is located over the portion between the drain 15 and the source 13.

The reset gate RG is formed over the portion between the photoelectric conversion portion 11 and the drain 12. In the example layout of FIG. 4, the drain 12 of the reset transistor RET is shared by the two adjoining pixel portions 100 (i.e., the pixel portion 100 being described and the adjacent pixel portion 100) and the reset gate RG also extends to the portion between the drain 12 and the photoelectric conversion portion 11 of the adjacent pixel portion 100.

The global interconnections extending in the row direction (i.e., read control lines rcg1 and rcg2, write control lines wcg1 and wcg2, and reset line RESET) are formed in a layer over the write control gates WCG1 and WCG2, the read control gates RCG1 and RCG2, and the reset gate RG with an insulating layer interposed in between.

The read control line rcg1 and the write control line wcg1 extend in the row direction alongside the top sideline of the pixel portion 100. The write control line wcg2, the read control line rcg2, and the reset line RESET extend in the row direction alongside the bottom sideline of the pixel portion 100.

The read control gate RCG1 extends to under the read control line rcg1 and is electrically connected to the read control line rcg1 there by a contact via 18. The write control gate WCG1 extends to under the write control line wcg1 and is electrically connected to the write control line wcg1 there by a contact via 17.

The read control gate RCG2 extends to under the read control line rcg2 and is electrically connected to the read control line rcg2 there by a contact via 19. The write control gate WCG2 extends to under the write control line wcg2 and is electrically connected to the write control line wcg2 there by a contact via 16.

The reset gate RG extends to under the reset line RESET and is electrically connected to the reset line RESET there by a contact via RGa.

An insulating layer is formed over the read control lines rcg1 and rcg2, the write control lines wcg1 and wcg2, and the reset line RESET, and the global interconnections extending in the column direction (i.e., column signal line OL, source line SL, and reset drain line RD) are formed on the insulating layer.

One column signal line OL and one source line SL are provided for each set of pixel portions 100 arranged in the column direction and one reset drain line RD is provided for each two sets of pixel portions 100 arranged in the column direction.

The column signal line OL projects to over the drain 14 and is electrically connected to the drain 14 there by a via 14a. The column signal line OL also projects to over the drain 15 and is electrically connected to the drain 15 there by a via 15a.

The source line SL projects to over the source 13 and is electrically connected to the source 13 there by a via 13a.

The reset drain line RD passes over the drain 12 and is electrically connected to the drain 12 by a contact via 12a.

In the example layout of FIG. 4, the drains of the write transistors WT1 and WT2 are omitted, that is, each of the write transistors WT1 and WT2 is a two-terminal MOS transistor whose source (also serves as the drain) is connected to the photoelectric conversion portion 11. A resistor, coil, capacitor, diode, etc. are known as two-terminal devices, and there are no active two-terminal devices that serve for switching or signal amplification.

It is common knowledge that a transistor as an active device that serves for pixel selection, resetting, signal writing, signal reading, or the like in a general solid-state imaging device does not function properly in the form of a two-terminal device. No one has ever made an attempt to use a transistor as a two-terminal device.

In the configuration of the solid-state imaging device 10 shown in FIGS. 2A and 2B, the write transistor WT1 and the read transistor RT1 share the floating gate FG1. Therefore, the write transistor WT1 is only required to serve for a single kind of operation of writing (charge injection into (writing to) the floating gate FG1) in which charge movement occurs only in one direction. Because of the common floating gate structure, the adjacent read transistor RT1 can read a signal. It has been found that there is no problem in operation even though the write transistor TR1 has the two-terminal structure. This is also true of the write transistor WT2.

In the solid-state imaging device 10, the degree of design freedom tends to be lowered because the plural charge storage portions need to be formed in each pixel portion 100. In this situation, the fact that each of the write transistors WT1 and WT2 has the two-terminal structure that consists of the write control gate and the source connected to the photoelectric conversion portion 11 is effective in terms of simplification of the configuration of the solid-state imaging device 10. In addition, in the example of FIG. 4, the read transistors RT1 and RT2 share the source and the two adjoining pixel portions 100 share the reset transistor RET. These features make it possible to reduce the size of each pixel portion 100 and the chip size and to realize increase in the number of pixels, miniaturization, etc. of the solid-state imaging device 10.

Returning to FIG. 1, the light source 1 is a light emitting unit capable of emitting plural kinds of light in a selective manner. The light source 1 includes an ordinary light unit which emits ordinary light that is necessary to take an object image as would be recognized through the naked eye and a special light unit which emits special light that is necessary to enable discrimination of a part that cannot be discriminated with ordinary light.

For example, the ordinary light unit includes a halogen lamp 1a which emits white light. Satisfactory results are obtained as long as the ordinary light unit can emit light that includes light components in red, green, and blue wavelength ranges. For example, the ordinary light unit may include LEDs which emit red light, green light, and blue light, respectively, at the same time.

The special light unit emits special light-1, 2 and 3 in different wavelength ranges, that is, special light having three emission wavelength ranges. The special light unit includes LEDs 1b, 1c, and 1d whose emission wavelengths can be set arbitrarily.

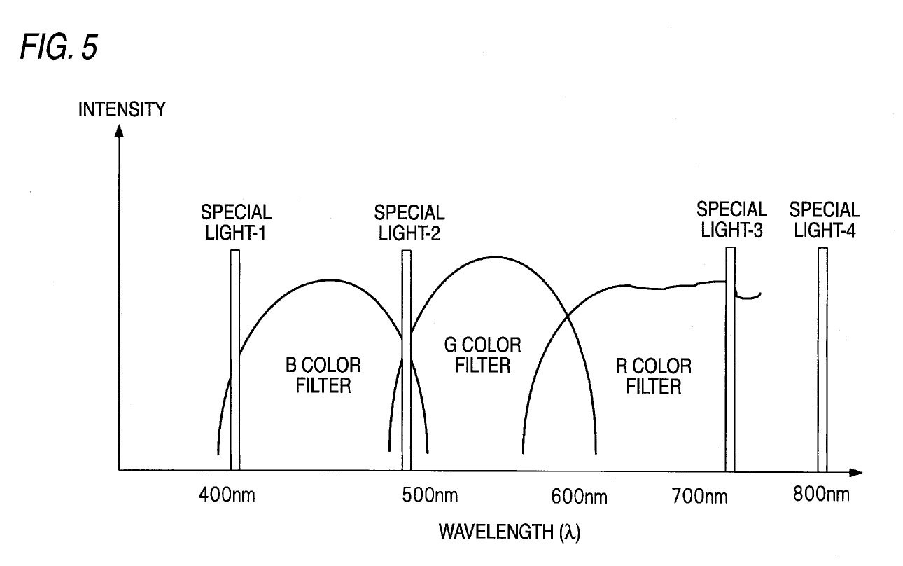

As shown in FIG. 5, the LED 1b emits special light-1 having an emission line at a particular wavelength that is located in the transmission wavelength range of the blue color filter. As shown in FIG. 5, the LED 1c emits special light-2 having an emission line at a particular wavelength that is located in the transmission wavelength range of the green color filter. As shown in FIG. 5, the LED 1d emits special light-3 having an emission line at a particular wavelength that is located in the transmission wavelength range of the red color filter.

The particular wavelengths of the special light-1, 2, and 3 may be determined in advance according to living body information to be detected by observation. Various wavelengths can be set such as a wavelength at which an object should be illuminated to clearly recognize presence/absence of redness (hemoglobin), a wavelength at which an object should be illuminated to clearly recognize occurrence/non-occurrence of autofluorescence, and a wavelength at which an object should be illuminated to clearly recognize deep blood vessels of an object.

There are objects which emit excitation-induced light having a different wavelength in response to reception of light having a particular wavelength. And there may occur a case that it is desired to observe an image of such excitation-induced light. Since light other than red, green, and blue light does not shine on the photoelectric conversion portion 11, the solid-state imaging device 10 can detect excitation-induced light if it has an emission wavelength in the red, green, or blue wavelength range. To detect excitation-induced light, it is necessary to emit special light having such an emission wavelength as to be able to cause an object to generate excitation-induced light. On the other hand, where light coming from an object has the same wavelength as special light, the emission wavelength of the special light can be set equal to a wavelength for detection.

For example, to detect light reflected from an object in response to reception of light having a wavelength 400 nm, the special light unit may be configured so as to be able to emit special light having a wavelength 400 nm. To detect light reflected from an object in response to reception of light having a wavelength 400 nm and light reflected from the object in response to reception of light having a wavelength 500 nm, the special light unit may be configured so as to be able to emit special light beams having wavelengths 400 nm and 500 nm. To detect excitation-induced light having a wavelength 680 nm generated by an object in response to reception of light having a wavelength 650 nm, light reflected from the object in response to reception of light having a wavelength 400 nm, and light reflected from the object in response to reception of light having a wavelength 500 nm, the special light unit may be configured so as to be able to emit special light beams having wavelengths 400 nm, 500 nm, and 650 nm.

Where the number of image data to be acquired is two (i.e., an image taken with white light and an image taken with special light), the emission wavelength of special light to be emitted from the special light unit may be set so that reflection light or excitation-induced light coming from an object in response to reception of the special light will have a particular wavelength that is located in one of the red, green, ad blue wavelength ranges. Where the number of image data to be acquired is three (i.e., an image taken with white light and two images taken with special light beams), the emission wavelengths of special light beams to be emitted from the special light unit may be set so that reflection light or excitation-induced light coming from an object in response to reception of the special light will have particular wavelengths that are located in two of the red, green, and blue wavelength ranges, respectively. Where the number of image data to be acquired is four (i.e., an image taken with white light and three images taken with special light beams), the emission wavelengths of special light beams to be emitted from the special light unit may be set so that reflection light or excitation-induced light coming from an object in response to reception of the special light beams will have particular wavelengths that are located in two of the red, green, and blue wavelength ranges, respectively.

The light source drive section 21, which drives the light source 1, selectively performs a drive for causing the ordinary light unit to emit ordinary light and a drive for causing the special light unit to emit special light (beams). The signal processing section 23 generates data by performing signal processing on imaging signals that are output from the solid-state imaging device 10. The generated image data is recorded in a recording medium or displayed on the display unit 22.

The system control section 24 supervises the entire endoscope apparatus. The manipulation unit 25 is an interface for various manipulations to be performed on the endoscope apparatus.

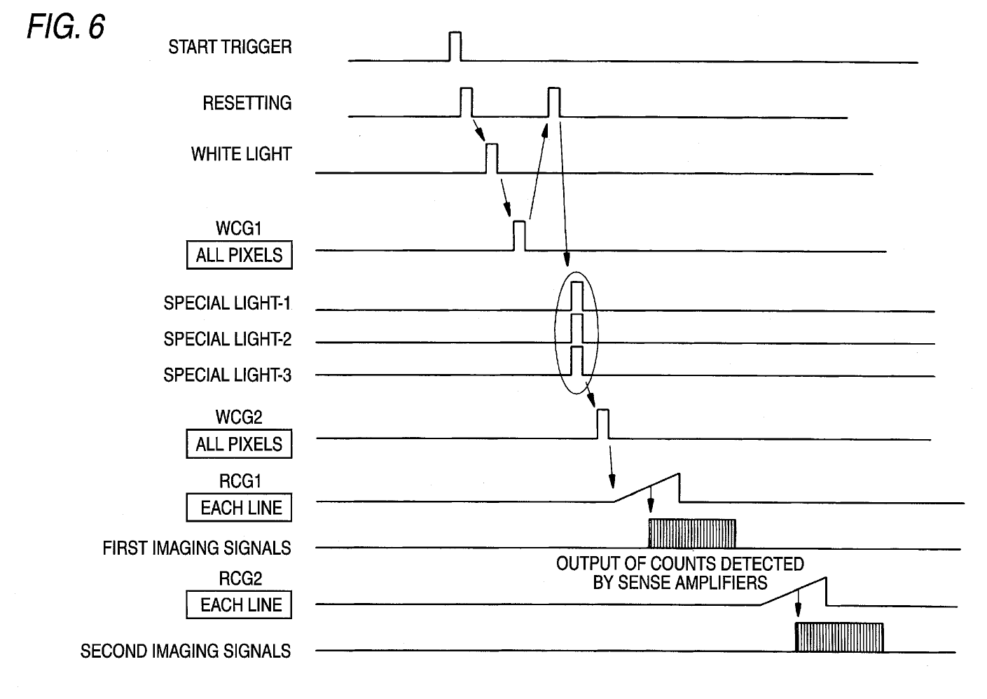

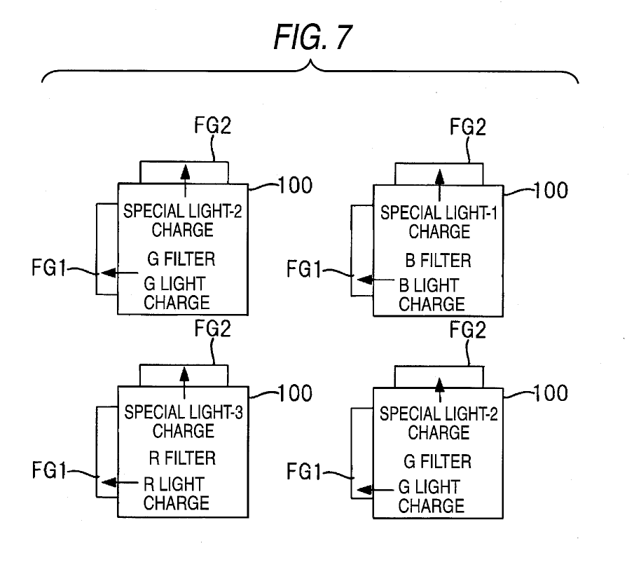

The operation of the above-configured endoscope apparatus will be described below. FIG. 6 is a timing chart showing an operation of the endoscope apparatus of FIG. 1. FIG. 7 is a schematic diagram showing the operation of the endoscope apparatus of FIG. 1.

When an instruction to shoot an object is issued by manipulation of the manipulation unit 25, the instruction is input to the system control section 24 and the system control section 24 gives a shooting instruction to the solid-state imaging device 10.

When the solid-state imaging device 10 receives the shooting instruction, triggered by the shooting instruction the control section 40 supplies reset pulses to the reset gates RG of the reset transistors RET of all the pixel portions 100. As a result, the unnecessary charges stored in the photoelectric conversion portions 11 are ejected to the drains of the reset transistors RET.

Upon completion of the resetting, the system control section 24 issues an instruction to the light source drive section 21 and thereby causes the halogen lamp 1a to emit white light. Although in FIG. 6 white light is emitted with a short delay from the supply of the reset pulses, it is preferable that white light be emitted simultaneously with the completion of the resetting.

The white light emission is performed for, for example, an exposure time that is set in the endoscope apparatus. During the white light emission period, in each pixel portion 100 having a red color filter of the solid-state imaging device 10, only red light of light coming from an object passes through the red color filter and enters the photoelectric conversion portion 11, where charge corresponding to the amount of the red light is generated and stored. In each pixel portion 100 having a green color filter of the solid-state imaging device 10, only green light of light coming from the object passes through the green color filter and enters the photoelectric conversion portion 11, where charge corresponding to the amount of the green light is generated and stored. In each pixel portion 100 having a blue color filter of the solid-state imaging device 10, only blue light of light coming from the object passes through the blue color filter and enters the photoelectric conversion portion 11, where charge corresponding to the amount of the blue light is generated and stored.

Upon termination of the exposure period, the control section 40 supplies write pulses to the write control gates WCG1 of all the pixel portions 100 and thereby causes the floating gates FG1 to store the charges that were generated by the photoelectric conversion portions 11 during the exposure period. The supply of write pulses may be started either simultaneously with the end of the exposure period or simultaneously with the start of the exposure period (finished simultaneously with the end of the exposure period).

As shown in FIG. 7, upon the supply of the write pulses, the charges generated in the respective pixel portions 100 (i.e., the charges generated by the red light, the charges generated by the green light, and the charges generated by the blue light) are stored in the floating gates FG1 of the respective pixel portions 100.

Upon completion of the charge storage in the floating gates FG1, the control section 40 again supplies reset pulses to the reset gates RG of the reset transistors RET of all the pixel portions 100. As a result, the residual charges remaining in the photoelectric conversion portions 11 without being injected into the floating gates FG1 are ejected to the drains of the reset transistors RET.

After completion of the second resetting, the system control section 24 issues an instruction to the light source drive section 21 to cause the special light unit to emit special light beams (special light-1, 2, and 3). Although in FIG. 6 special light beams are emitted after a short delay from the supply of the reset pulses, it is preferable that special light beams be emitted simultaneously with the completion of the resetting.

The special light emission is performed for, for example, an exposure time that is set in the endoscope apparatus. During the special light emission period, in each pixel portion 100 having a red color filter of the solid-state imaging device 10, only reflection light or excitation-induced light corresponding to the special light-3 of light coming from an object passes through the red color filter and enters the photoelectric conversion portion 11, where charge is generated and stored. In each pixel portion 100 having a green color filter of the solid-state imaging device 10, only reflection light or excitation-induced light corresponding to the special light-2 of light coming from the object passes through the green color filter and enters the photoelectric conversion portion 11, where charge is generated and stored. In each pixel portion 100 having a blue color filter of the solid-state imaging device 10, only reflection light or excitation-induced light corresponding to the special light-1 of light coming from the object passes through the blue color filter and enters the photoelectric conversion portion 11, where charge is generated and stored.

Upon termination of the exposure period, the control section 40 supplies write pulses to the write control gates WCG2 of all the pixel portions 100 and thereby causes the floating gates FG2 to store the charges that were generated by the photoelectric conversion portions 11 during the exposure period. The storing of charges may be started either simultaneously with the end of the exposure period or simultaneously with the start of the exposure period (finished simultaneously with the end of the exposure period).

As shown in FIG. 7, upon the supply of the write pulses, the charges generated in the respective pixel portions 100 (i.e., the charges generated by the special light-1, the charges generated by the special light-2, and the charges generated by the special light-3) are stored in the floating gates FG2 of the respective pixel portions 100.

In the solid-state imaging device 10, the write control gates WCG1 and WCG2 are connected to the different control lines wcg1 and wcg2. Therefore, as described above, charges generated by each photoelectric conversion portion 11 by two times of exposures can be stored in the different floating gates FG1 and FG2.

After completion of the charge storage in the floating gates FG2, the control section 40 sets the drain potentials of the read transistors RT1 of the pixel portions 100 of the first line at Vr (<Vcc) and starts to apply ramp voltages to the read control gates RCG1 of the pixel portions 100 of the first line (after the start of application of the ramp voltages, the counts are incremented from an initial value (e.g., “0”), for example). Counts corresponding to values of ramp voltages that are applied when the drain potentials of the read transistors TR1 of the first line drop are latched in the reading circuits 20, respectively, and the latched counts are output from the output amplifier 60 as imaging signals. The control section 40 performs the same drive operation for the second and following lines and thereby causes output of first imaging signals (R signals, G signals, and B signals) corresponding to the amounts of charges stored in the floating gates FG1 of all the lines.

Then, the control section 40 sets the drain potentials of the read transistors RT2 of the pixel portions 100 of the first line at Vr (<Vcc) and starts to apply ramp voltages to the read control gates RCG2 of the pixel portions 100 of the first line (after the start of application of the ramp voltages, the counts are incremented from an initial value (e.g., “0”), for example). Counts corresponding to values of ramp voltages that are applied when the drain potentials of the read transistors TR2 of the first line drop are latched in the reading circuits 20, respectively, and the latched counts are output from the output amplifier 60 as imaging signals. The control section 40 performs the same drive operation for the second and following lines and thereby causes output of second imaging signals (special light-1 signals, special light-2 signals, and special light-3 signals) corresponding to the amounts of charges stored in the floating gates FG2 of all the lines.

After the output of the second imaging signals, the control section 40 sets the potentials of the write control gates WCG1 and WCG2 and the read control gates RCG1 and RCG2 of all the pixel portions 100 at −Vcc and sets the potential of the semiconductor substrate at Vcc. As a result, the charges stored in the floating gates FG1 and FG2 are drawn into the semiconductor substrate and thereby erased.

The above operation is performed in one frame period.

Since the first imaging signals are the same as imaging signals that are output from a Bayer-type solid-state imaging device, ordinary RGB color image data can be generated by causing the signal processing circuit 23 to perform known signal processing. A color image as would be recognized by observing the same object through the naked eye can be reproduced on the display unit 22 on the basis of the RGB color image data.

The second imaging signals include signals corresponding to the special light-1, signals corresponding to the special light-2, and signals corresponding to the special light-3. Therefore, the signal processing section 23 generates one image data from the signals corresponding to the special light-1, one image data from signals corresponding to the special light-2, and one image data from and the signals corresponding to the special light-3. Based on these image data, living body information of the object that is hard to recognize through the naked eye can be reproduced in an emphasized manner.

As described above, in the endoscope apparatus of FIG. 1, it is not necessary to read out signals corresponding to generated charges every time an exposure is performed. That is, sets of signals can be read out together after plural times of exposures. As a result, the intervals between plural times of exposures can be shortened and shooting operations using plural light sources can be performed successively in a short time. This makes it possible to take images of approximately the same object under different sets of conditions. For example, an image that enables recognition of presence/absence of redness, an image that enables recognition of occurrence/non-occurrence of autofluorescence, and an image that enables recognition of a state of deep blood vessels can be obtained together with an ordinary image for the same object. This makes it possible to increase the diagnosis accuracy of an endoscope examination. Furthermore, it is possible to display, in an emphasized manner, images obtained by different wavelengths, pulse widths, or intensities. This increases the diagnosis accuracy to a very large extent.

The endoscope apparatus of FIG. 1 is configured in such a manner that shooting is performed two times in a one-frame period (i.e., a period for acquiring image data of one frame) and sets of imaging signals are read out after completion of the two times of shooting. To perform shooting two times in a one-frame period, it is necessary to shorten the interval between the two times of shooting. In general solid-state imaging devices, it is necessary to read out imaging signals every time shooting is completed. Therefore, to shorten the shooting interval, it is necessary to read out imaging signal at high speed, which increases the heat generation amount of the device accordingly. Furthermore, since the exposure time needs to be shortened, the problem of sensitivity reduction becomes more serious. In endoscope apparatus, it is necessary to minimize the heat generation in the tip portion which is inserted into a body of a subject. If the heat generation amount is large, the tip portion needs to be provided with a cooling mechanism or the like. This obstructs miniaturization of the tip portion. In contrast, in the endoscope apparatus of FIG. 1, the shooting interval can be shortened without the need for reading out image signals at high speed. Therefore, the heat generation in the tip portion can be suppressed and hence the tip portion can be miniaturized.

In the endoscope apparatus of FIG. 1, a drive for ejecting the charges stored in the photoelectric conversion portions 11 to the reset drains is performed before charges are stored in the floating gates FG2. This prevents mixing of charges when exposures are performed using different light sources. As a result, color contamination can be prevented and the image quality can be increased.

In the endoscope apparatus of FIG. 1, imaging signals produced by using white light, imaging signals produced by using special light-1, imaging signals produced by using special light-2, and imaging signals produced by using special light-3 can be read out independently of each other. As a result, for example, the probability of occurrence of a false color can be made lower and the signal processing amount can be made smaller than in the case of acquiring imaging signals by emitting white light and special light simultaneously and extracting white color components and a special light component separately from the imaging signals. This enables increase in image quality and cost reduction.

Although in the above description each of the first charge storage portion and the second charge storage portion is composed of two MOS transistors, that is, the write transistor WT and the read transistor RT, each of them may be formed by a single transistor.

For example, referring to FIG. 3, a configuration is possible in which the read transistors RT1 and RT2 are omitted and the write transistors WT1 and WT2 are provided with drains to which the reading circuit 20 is connected by the column signal line OL. In this configuration, a first imaging signal is read out by setting the drain potential of the write transistor WT1 at Vr and applying a ramp voltage to the read control gate WCG1 and a second imaging signal is read out by setting the drain potential of the write transistor WT2 at Vr and applying a ramp voltage to the read control gate WCG2.

Where each charge storage portion is implemented as a single transistor, the transistor may have a structure other than the MOS structure. For example, the transistor may have an MNOS structure in which the floating gate FG1 is a nitride layer and the write control gate WCG1 is formed directly on the nitride layer or a MONOS structure in which the floating gate FG1 is a nitride layer. In either case, the nitride layer functions as a charge storage region.

An example configuration in which each of the first charge storage portion and the second charge storage portion is implemented as a single transistor will be described below.

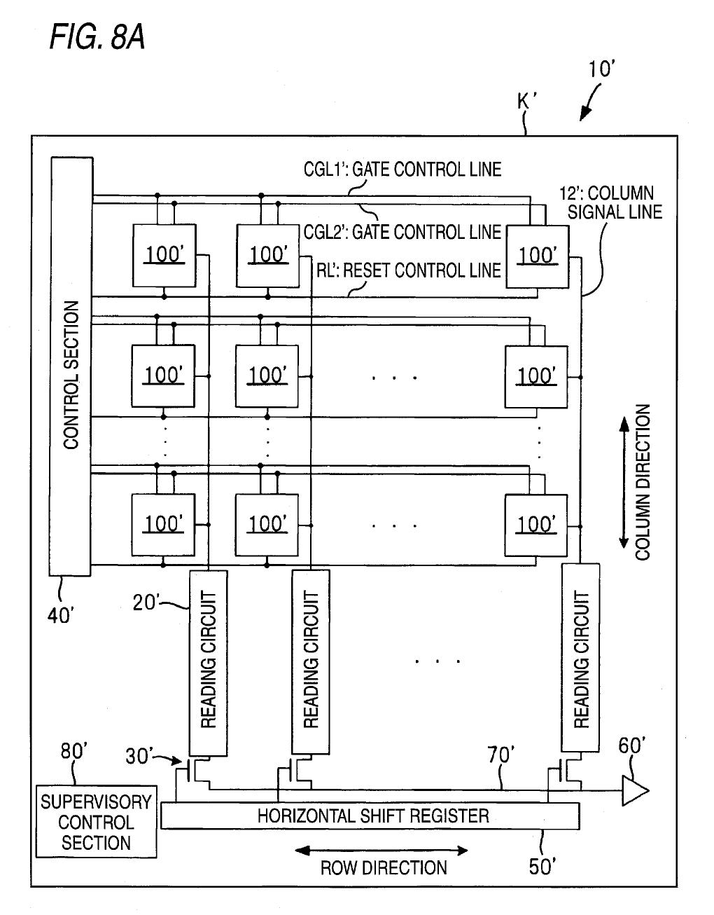

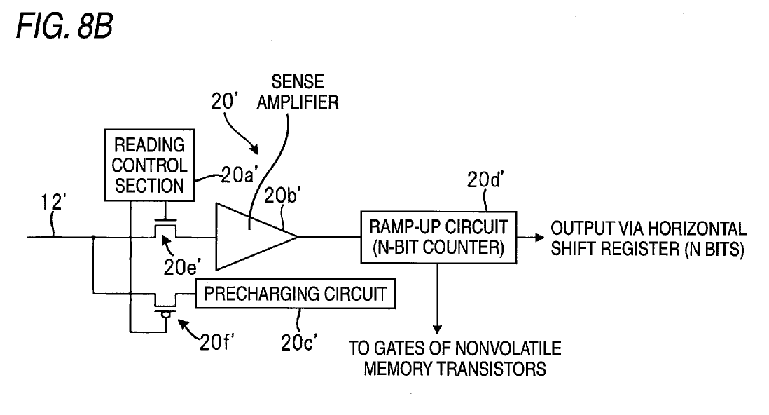

FIG. 8A is a schematic plan view showing a general configuration of the whole of a solid-state imaging device according to another embodiment of the invention, and FIG. 8B is a block circuit diagram of each reading circuit of the solid-state imaging device of FIG. 8A. The solid-state imaging device 10′ of FIG. 8A is provided with pixel portions 100′, reading circuits 20′, an output circuit (transistors 30′, signal line 70′, horizontal shift register 50′, and output amplifier 60′), a control section 40′, and a supervisory control section 80′.

The plural pixel portions 100′ are arranged in a column direction of a semiconductor substrate K′ and a row direction that is perpendicular to the column direction so as to form a two-dimensional array (in this example, a square lattice).

The reading circuits 20′ are provided for the respective sets of pixel portions 100′ arranged in the column direction, and serve to read out imaging signals from the respective sets of pixel portions 100′.

The output amplifier is a circuit for outputting imaging signals of pixel portions 100′ of one row that are read out by the reading circuits 20′.

The control section 40′ controls the pixel portions 100′.

The supervisory control section 80′ supervises the entire solid-state imaging device 10′. The solid-state imaging device 10′ operates as the supervisory control section 80′ supervises the individual sections under the control of a system control section of an imaging apparatus incorporating it.

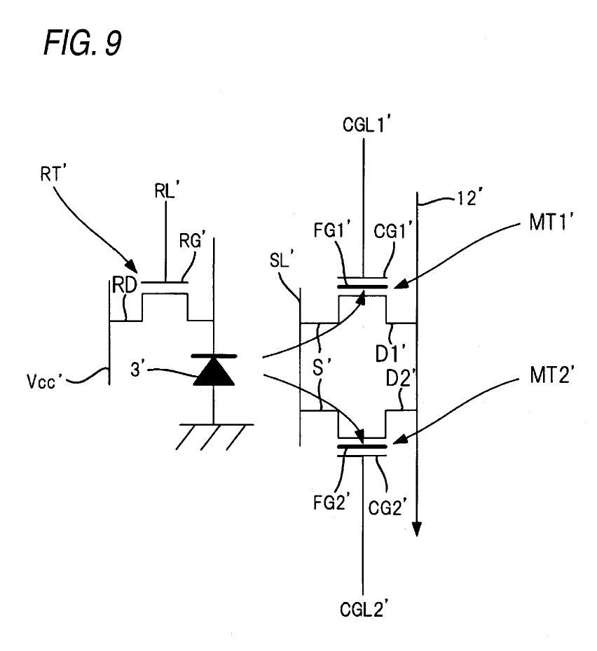

FIG. 9 is an equivalent circuit diagram of each pixel portion of the solid-state imaging device 10′ of FIG. 8A. As shown in FIG. 9, each pixel portion 100′ is provided with a photoelectric conversion portion 3′, nonvolatile memory transistors MT1′ and MT2′, and a reset transistor RT′.

The photoelectric conversion portion 3′ is formed in the semiconductor substrate K′. The nonvolatile memory transistor MT1′ has a MOS transistor structure which includes a floating gate FG1′ which is a charge storage region formed on the semiconductor substrate K′ and a control gate CG1′ which is a gate electrode. The nonvolatile memory transistor MT2′ has a MOS transistor structure which includes a floating gate FG2′ which is a charge storage region formed on the semiconductor substrate K′ and a control gate CG2′ which is a gate electrode. The reset transistor RT′ serves to reset the charge stored in the photoelectric conversion portion 3′. The nonvolatile memory transistors MT1′ and MT2′ function as charge storage regions a selected one of which can store charge generated by the photoelectric conversion portion 3′.

The outputs (drain regions D1′ and D2′) of the nonvolatile memory transistors MT1′ and MT2′ are both connected to a column signal line 12′ which is one of output signal lines provided for the respective sets of pixel portions 100′ arranged in the column direction. The reading circuit 20′ is connected to the column signal line 12′. The sources S′ of the nonvolatile memory transistors MT1′ and MT2′ are both connected to a source line SL′ which is one of the source lines SL′ provided for the respective sets of pixel portions 100′ arranged in the column direction.

The reset transistor RT′ has a MOS structure having a reset drain RD′, the photoelectric conversion portion 3′ which functions as a source region, and a reset gate RG′ which is a gate electrode. A reset power line Vcc′ for supplying a reset voltage is connected to the reset drain RD′.

A gate control line CGL1′ which is one of the gate control lines CGL1′ provided for the respective lines, that is, the respective sets of pixel portions 100′ arranged in the row direction, is connected to the control gate CG1′ of the nonvolatile memory transistor MT1′. The gate control lines CGL1′ for the respective lines are connected to the control section 40′, whereby voltages can be supplied to the control gates CG1′ independently on a line-by-line basis.

A gate control line CGL2′ which is one of the gate control lines CGL2′ provided for the respective lines is connected to the control gate CG2′ of the nonvolatile memory transistor MT2′. The gate control lines CGL2′ for the respective lines are connected to the control section 40′, whereby voltages can be supplied to the control gates CG2′ independently on a line-by-line basis.

A reset control line RL′ which is one of the reset control lines RL′ provided for the respective lines is connected to the reset gate RG′ of the reset transistor TR′. The reset control lines RL′ provided for the respective lines are connected to the control section 40′, whereby voltages can be supplied to the reset gates RG′ independently on a line-by-line basis. When a reset pulse is applied to the reset gate RG′ from the control section 40′ via the reset control line RL′, the reset transistor RT′ is turned on and the charge stored in the photoelectric conversion portion 3′ is ejected to the drain RD′ of the reset transistor RT′.

As shown in FIG. 8B, each reading circuit 20′ is provided with a reading control section 20a′, a sense amplifier 20b′, a precharging circuit 20c′, a ramp-up circuit 20d′, and transistors 20e′ and 20f′.

The reading control section 20a′ on/off-controls the transistors 20e′ and 20f′. The precharging circuit 20c′ is a circuit for precharging the column signal line 12′ by supplying a prescribed voltage to it. Monitoring the voltage of the column signal line 12′, the sense amplifier 20b′ detects a change in the voltage and informs the ramp-up circuit 20d′ of that fact. For example, the sense amplifier 20b′ detects a drop from the drain voltage value produced by the precharging by the precharging circuit 20c′ and inverts its output.

Incorporating an N-bit counter (e.g., N=8 to 12), the ramp-up circuit 20d′ supplies a gradually increasing or decreasing ramp voltage to the control gate CG1′ or CG2′ of the pixel portion 100′ via the control section 40 and outputs a count (a combination of 1s and 0s numbering N together) that corresponds to the ramp voltage value.

If the voltage of the control gate CG1′ exceeds the threshold voltage of the nonvolatile memory transistor MT1′ in a state that the column signal line 12′ is precharged, the nonvolatile memory transistor MT1′ is turned on and the potential of the column signal line 12′ drops which has been precharged. The sense amplifier 20b′ detects this event and outputs an inverted signal. The ramp-up circuit 20d′ latches a count corresponding to a ramp voltage value being generated at the time point of reception of the inverted signal. In this manner, a change in the threshold voltage (i.e., a change with respect to a threshold voltage of a case that no charge is stored in the floating gate FG1′) of the nonvolatile memory transistor MT1′ can be read out as a signal.

If the voltage of the control gate CG2′ exceeds the threshold voltage of the nonvolatile memory transistor MT2′ in a state that the column signal line 12′ is precharged, the nonvolatile memory transistor MT2′ is turned on and the potential of the column signal line 12′ drops which has been precharged. The sense amplifier 20b′ detects this event and outputs an inverted signal. The ramp-up circuit 20d′ latches a count corresponding to a ramp voltage value being generated at the time point of reception of the inverted signal. In this manner, a change in the threshold voltage (i.e., a change with respect to a threshold voltage of a case that no charge is stored in the floating gate FG2′) of the nonvolatile memory transistor MT2′ can be read out as a signal.

When one horizontal selection transistor 30′ is selected by the horizontal shift register 50′, a count that is latched by the ramp-up circuit 20d′ connected to the selected horizontal selection transistor 30′ is output to the signal line 70′ and output from the output amplifier 60′ as an imaging signal.

The method for reading out a change in the threshold voltage of the nonvolatile memory transistor MT1′ or MT2′ as a signal is not limited to the above method. For example, a drain current of the nonvolatile memory transistor MT1′ that occurs when a prescribed voltage is applied between the control gate CG1′ and the drain region D1′ of the nonvolatile memory transistor MT1′ and a drain current of the nonvolatile memory transistor MT2′ that occurs when a prescribed voltage is applied between the control gate CG2′ and the drain region D2′ of the nonvolatile memory transistor MT2′ may be read out as signals.

The control section 40′ performs a drive for injecting charge generated by the photoelectric conversion portion 3′ into the floating gate FG1′ or FG2′ and storing the charge there by controlling the nonvolatile memory transistors MT1′ and MT2′ independently. In the nonvolatile memory transistor MT1′ (or MT2′), charge generated by the photoelectric conversion portion 3′ is injected into the floating gate FG1′ (or FG2′) by Fowler-Nordheim (F-N) tunneling injection using F-N tunneling current, direct tunneling electron injection, hot electron injection, or the like by applying a write pulse to the control gate CG1′ (or CG2′) and stored there.

Furthermore, the control section 40′ performs a reset drive for rendering empty the photoelectric conversion portion 3′ of each pixel portion 100′ by ejecting, to the outside, charge generated by and stored in the photoelectric conversion portion 3′ and a charge erasing drive for erasing the charge stored in the floating gate FG1′ or FG2′ by ejecting it to the semiconductor substrate K′.

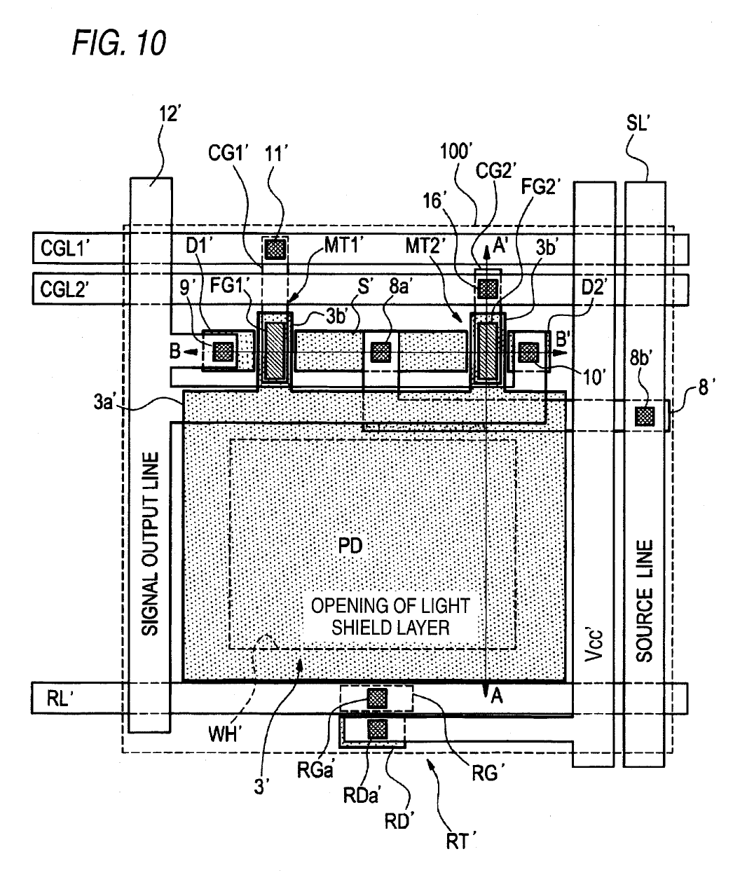

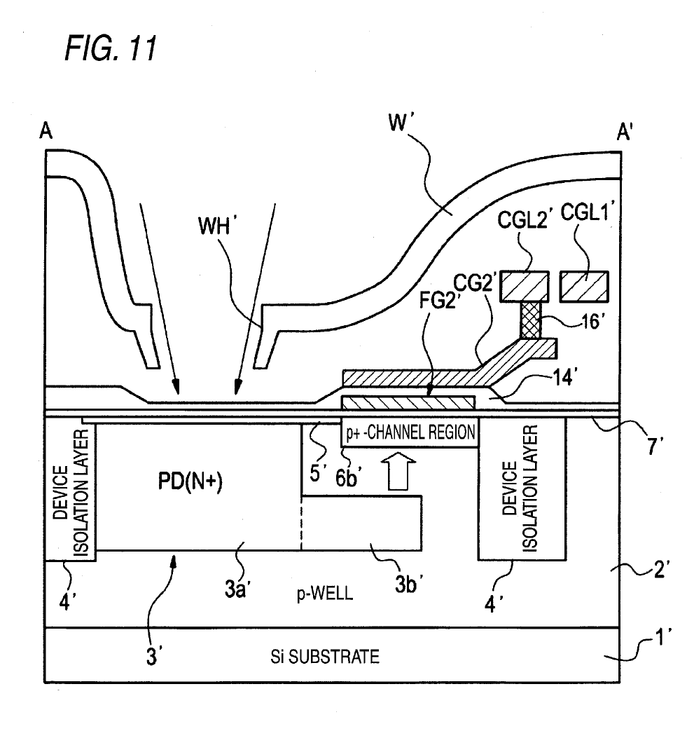

FIG. 10 is a schematic plan view showing an example layout of each pixel portion 100′ of the solid-state imaging device 10′ of FIG. 8A. FIG. 11 is a schematic sectional view of the pixel portion 100′ taken along line B-B′ in FIG. 10.

As shown in FIG. 11, the photoelectric conversion portion 3′ has an n-type impurity region formed in a p-well layer 2′ of an n-type silicon substrate 1′. The photoelectric conversion function is realized by the pn junction of the n-type impurity region and the p-well layer 2′. The photoelectric conversion portion 3′ is what is called a buried photodiode in which a p-type impurity layer 5′ for complete depletion and dark current suppression is formed adjacent to the surface. The n-type silicon substrate 1′ and the p-well layer 2′ constitute the above-mentioned semiconductor substrate K′.

Adjoining pixel portions 100′ are isolated from each other by a device isolation layer 4′ formed in the p-well layer 2′. Example device isolation methods are the LOCOS (local oxidation of silicon), the STI (shallow trench isolation) method, and a method using high-concentration impurity ion implantation.

The source region S′ of the nonvolatile memory transistor MT1′ is an n-type impurity region which is spaced from the photoelectric conversion portion 3′ in the column direction. The drain region D1′ of the nonvolatile memory transistor MT1′ is an n-type impurity region which is spaced from the source region S′ in the row direction. A channel region 6a′ which is a p-type impurity region is formed between the source region S′ and the drain region D1′. The floating gate FG1′ is formed over the channel region 6a′ with an insulating layer 7′ interposed in between, and the control gate CG1′ is formed over the floating gate FG1′ with an insulating layer 14′ interposed in between. The channel region 6a′ is a region where carriers flow according to a voltage that is applied to the control gate CG1′. Although in this example the channel region 6a′ is formed by implanting a p-type impurity into the region between the source region S′ and the drain region D1′, this region may be left as it is (as part of the p-well layer 2′).

The drain region D2′ of the nonvolatile memory transistor MT2′ is an n-type impurity region which is spaced from the source region S′ in the row direction. A channel region 6b′ which is a p-type impurity region is formed between the source region S′ and the drain region D2′. The floating gate FG2′ is formed over the channel region 6b′ with the insulating layer 7′ interposed in between, and the control gate CG2′ is formed over the floating gate FG2′ with the insulating layer 14′ interposed in between. The channel region 6b′ is a region where carriers flow according to a voltage that is applied to the control gate CG2′. Although in this example the channel region 6b′ is formed by implanting a p-type impurity into the region between the source region S′ and the drain region D2′, this region may be left as it is (as part of the p-well layer 2′).

For example, the conductive material of the control gates CG1′ and CG2′ may be polysilicon or doped polysilicon which is doped heavily with phosphorus (P), arsenic (As), or boron (B). As a further alternative, the conductive material may be silicide which is a combination of silicon and any of various metals such as titanium (Ti) and tungsten (W) or the control gates CG1′ and CG2′ may have a salicide (self-aligned silicide) structure. The floating gates FG1′ and FG2′ may be made of the same conductive material as the control gates CG1′ and CG2′.

In the example layout of FIG. 10, the source region S′ and the drain regions D1′ and D2′ are aligned in the row direction and the long and narrow floating gates FG1′ and FG2′ and control gates CG1′ and CG2′ extend in the column direction between the source region S′ and the drain regions D1′ and D2′. The control gate CG1′ extends to under a gate control line CGL1′ which is an aluminum interconnection extending in the row direction, and is connected to the gate control line CGL1′ there by a contact 11′ which is made of aluminum or the like.

The control gate CG2′ extends to under a gate control line CGL2′ which is an aluminum interconnection extending in the row direction, and is connected to the gate control line CGL2′ there by a contact 16′ which is made of aluminum or the like.

Parts of the column signal line 12′ which is an aluminum interconnection extending in the column direction extend to over the respective drain regions D1′ and D2′, and are electrically connected to the drain regions D1′ and D2′ by contacts 9′ and 10′, respectively, which are made of aluminum or the like.

A contact 8a′ made of aluminum or the like is formed on the source region S′, and an interconnection 8′ is connected to the contact 8a′. The interconnection 8′ goes under the reset power line Vcc′ which is an aluminum interconnection extending in the column direction, and extends to under the source line SL′. The interconnection 8′ and the source line SL′ are electrically connected to each other by a contact 8b′ which are made of aluminum or the like. The source lines SL′ are provided for the respective sets of pixel portions 100′ arranged in the column direction, and are supplied with a prescribed potential (e.g., a ground potential).

The reset transistor RT′ has a MOS transistor structure having the photoelectric conversion portion 3′ which functions as a source region, the drain region RD′ which is an n-type impurity region spaced from the photoelectric conversion portion 3′ in the column direction, and the reset gate RG′ which is provided over the portion of the semiconductor substrate K′ which is located between the photoelectric conversion portion 3′ and the drain region RD′ with the insulating layer 7′ interposed in between.

In the example layout of FIG. 10, the reset gate RG′ is disposed under the reset control line RL′ which is an aluminum interconnection extending in the row direction, and is connected to the reset control line RL′ there by a contact RGa′ which is made of aluminum or the like.

Part of the reset power line Vcc′ extends to over the drain region RD′ and is electrically connected to the drain region RD′ by a contact RDa′ which is made of aluminum or the like. The reset power lines Vcc′ are provided to the respective sets of pixel portions 100′ arranged in the column direction, and is supplied with a prescribed power source voltage.

The manner of arrangement of the reset transistor RT′ and the nonvolatile memory transistors MT1′ and MT2′ is not limited to the one shown in FIG. 10, and they may be arranged as appropriate according to available spaces.

As for the positional relationships between the various interconnections, the source line SL′, the reset power line Vcc′, and the column signal line 12′ are formed in the layer that is located above the layer in which the gate control line CGL1′ and CGL2′, the reset control line RL′, and the interconnection 8′.

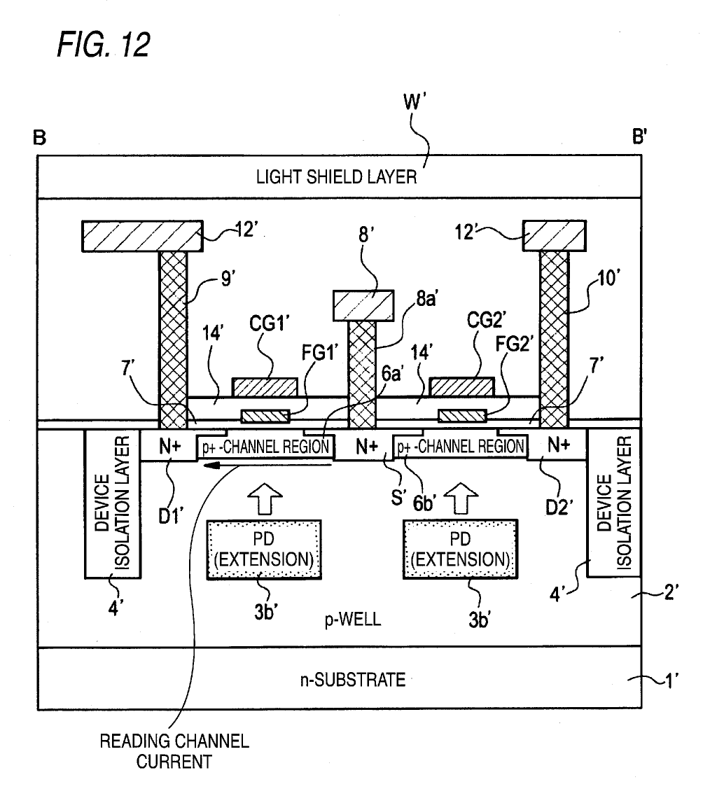

The pixel portion 100′ is configured in such a manner that a light shield layer W′ which is made of tungsten, for example, prevents light from shining on the area other than part of the photoelectric conversion portion 3′. As shown in FIGS. 11 and 12, the light shield layer W′ having an opening WH′ over the part of the photoelectric conversion portion 3′ is formed over the semiconductor substrate K′ (i.e., above the source line SL′, the reset power line Vcc′, and the column signal line 12′).

In the solid-state imaging device 10′, to increase the efficiency of charge injection into the floating gates FG1′ and FG2′, the photoelectric conversion portion 3′ not only exists under the opening WH′ of the light shield layer W′ but also extends to under the channel regions 6a′ and 6b′ of the nonvolatile memory transistors MT1′ and MT2′ (see FIGS. 11 and 12).

As shown in FIGS. 11 and 12, the photoelectric conversion portion 3′ consists of a main body 3a′ which is located under the opening WH′ and extensions 3b′ which extend to under the channel regions 6a′ and 6b′, respectively. Although a boundary line (broken line) is drawn in FIG. 11, this is just for the sake of description and no such boundary exists actually.

The main body 3a′ is the portion which is formed under the opening WH′ to receive light. The extensions 3b′ are the portions which extend to under the channel regions 6a′ and 6b′ of the nonvolatile memory transistors MT1′ and MT2′ in the p-well layer 2′. In a plan view, the extensions 3b′ extend in the column direction from those portions of the main body 3a′ which are opposed to the channel regions 6a′ and 6b′ to the channel regions 6a′ and 6b′. That is, the photoelectric conversion portion 3′ is formed in such a manner that in the plan-view areas where the nonvolatile memory transistors MT1′ and MT2′ and the reset transistor RT′ are formed, the photoelectric conversion portion 3′exists only under the channel regions 6a′ and 6b′ of the nonvolatile memory transistors MT1′ and MT2′. Alternatively, the extension 3b′ may be formed in such a manner that the photoelectric conversion portion 3′ exists not only under the channel regions 6a′ and 6b′ but also under the entire nonvolatile memory transistors MT1′ and MT2′.

The channel region 6a′ (or 6b′) is located directly under the control gate CG1′ (or CG2′) and the floating gate FG1′ (or FG2′). Therefore, forming the photoelectric conversion portion 3′ in such a manner that it extends to under the channel region 6a′ (or 6b′) (preferably, to the entire portions where the channel region 6a′ (or 6b′) exists in a plan view) allows an electric field to be developed approximately perpendicularly to the floating gate FG1′ (or FG2′) by a voltage (CG voltage) applied to the control gate CG1′ (or CG2′) in injecting charge into the floating gate FG1′ (or FG2′) from the photoelectric conversion portion 3′ by F-N tunneling injection or direct tunnel injection. This facilitates acceleration of charge moving from the photoelectric conversion portion 3′ toward the floating gate FG1′ (or FG2′). This makes it possible to cause the tunneling effect at a low CG voltage.

In the solid-state imaging device 10′, since the photoelectric conversion portion 3′ extends to under the channel region 6a′ (or 6b′) while securing the channel region 6a′ (or 6b′), there is no limitation on the size of the overlap between the photoelectric conversion portion 3′ and the control gate CG1′ (or CG2′) and an electric field can be developed approximately perpendicularly to the floating gate FG1′ (or FG2′). As a result, tunneling current can be generated efficiently.

The lengths of the photoelectric conversion portion 3′ parallel with the substrate surface can be controlled by controlling a mask pattern used in ion implantation, and its length in the direction perpendicular to the substrate surface can be controlled by controlling the ion implantation energy. The photoelectric conversion portion 3′ which consists of the main body 3a′ and the extensions 3b′ can be formed in these manners.

Next, a description will be made of the operation of an endoscope apparatus shown in FIG. 1 which incorporates the above-configured solid-state imaging device 10′.

When an instruction to shoot an object is issued by manipulation of the manipulation unit 25, the instruction is input to the system control section 24 and the system control section 24 gives a shooting instruction to the solid-state imaging device 10′.

When the solid-state imaging device 10′ receives the shooting instruction, triggered by the shooting instruction the control section 40′ supplies reset pulses to the reset gates RG′ of the reset transistors RET′ of all the pixel portions 100′. As a result, the unnecessary charges stored in the photoelectric conversion portions 3′ are ejected to the drains of the reset transistors RET′.

Upon completion of the resetting, the system control section 24 issues an instruction to the light source drive section 21 and thereby causes the halogen lamp 1a to emit white light.

The white light emission is performed for, for example, an exposure time that is set in the endoscope apparatus. During the white light emission period, in each pixel portion 100′ having a red color filter of the solid-state imaging device 10′, only red light of light coming from an object passes through the red color filter and enters the photoelectric conversion portion 3′, where charge corresponding to the amount of the red light is generated and stored. In each pixel portion 100′ having a green color filter of the solid-state imaging device 10′, only green light of light coming from the object passes through the green color filter and enters the photoelectric conversion portion 3′, where charge corresponding to the amount of the green light is generated and stored. In each pixel portion 100′ having a blue color filter of the solid-state imaging device 10′, only blue light of light coming from the object passes through the blue color filter and enters the photoelectric conversion portion 3′, where charge corresponding to the amount of the blue light is generated and stored.

Upon termination of the exposure period, the control section 40′ supplies write pulses to the control gates CG1′ of all the pixel portions 100′ and thereby causes the floating gates FG1′ to store the charges that were generated by the photoelectric conversion portions 3′ during the exposure period. The supply of write pulses may be started either simultaneously with the end of the exposure period or simultaneously with the start of the exposure period (finished simultaneously with the end of the exposure period).

Upon the supply of the write pulses, the charges generated in the respective pixel portions 100′ (i.e., the charges generated by the red light, the charges generated by the green light, and the charges generated by the blue light) are stored in the floating gates FG1′ of the respective pixel portions 100′.

Upon completion of the charge storage in the floating gates FG1′, the control section 40′ again supplies reset pulses to the reset gates RG′ of the reset transistors RET′ of all the pixel portions 100′. As a result, the residual charges remaining in the photoelectric conversion portions 3′ without being injected into the floating gates FG1′ are ejected to the drains of the reset transistors RET′.

After completion of the second resetting, the system control section 24 issues an instruction to the light source drive section 21 to cause the special light unit to emit special light beams (special light-1, 2, and 3).

The special light emission is performed for, for example, an exposure time that is set in the endoscope apparatus. During the special light emission period, in each pixel portion 100′ having a red color filter of the solid-state imaging device 10′, only reflection light or excitation-induced light corresponding to the special light-3 of light coming from an object passes through the red color filter and enters the photoelectric conversion portion 3′, where charge is generated and stored. In each pixel portion 100′having a green color filter of the solid-state imaging device 10′, only reflection light or excitation-induced light corresponding to the special light-2 of light coming from the object passes through the green color filter and enters the photoelectric conversion portion 3′, where charge is generated and stored. In each pixel portion 100′ having a blue color filter of the solid-state imaging device 10′, only reflection light or excitation-induced light corresponding to the special light-1 of light coming from the object passes through the blue color filter and enters the photoelectric conversion portion 3′, where charge is generated and stored.

Upon termination of the exposure period, the control section 40′ supplies write pulses to the control gates CG2′ of all the pixel portions 100′ and thereby causes the floating gates FG2′ to store the charges that were generated by the photoelectric conversion portions 3′ during the exposure period. The storing of charges may be started either simultaneously with the end of the exposure period or simultaneously with the start of the exposure period (finished simultaneously with the end of the exposure period).

Upon the supply of the write pulses, the charges generated in the respective pixel portions 100′ (i.e., the charges generated by the special light-1, the charges generated by the special light-2, and the charges generated by the special light-3) are stored in the floating gates FG2′ of the respective pixel portions 100′.

In the solid-state imaging device 10′, the control gates CG1′ and CG2′ are connected to the different control lines CGL1′ and CGL2′. Therefore, as described above, charges generated by each photoelectric conversion portion 3′ by two times of exposures can be stored in the different floating gates FG1′ and FG2′.

After completion of the charge storage in the floating gates FG2′, the reading control sections 20a′ turn on the transistors 20f′ to precharge the column signal lines 12′. Then, the reading control sections 20a′ turn on the transistors 20e′ to establish electrical continuity between the column signal lines 12′ and the sense amplifiers 20b′, respectively. In this state, the ramp-up circuits 20d′ start to apply ramp voltages (Vth reading voltages) to the control gates CG1′ of the pixel portions 100′ of the first line (after the start of application of the ramp voltages, the counts are incremented from an initial value (e.g., “0”)). Counts corresponding to values of ramp voltages that are applied when the drain potentials of the nonvolatile memory transistors MR1′ of the pixel portions 100′ of the first line drop are latched in the reading circuits 20′, respectively, and the latched counts are output from the output amplifier 60′ via the signal line 70′ under the control of the horizontal shift register 50′ as imaging signals that were generated by the photoelectric conversion portions 3′ during the exposure period when the white light was emitted and were stored there. After the output of the imaging signals, the transistors 20f are turned off, the application of the ramp voltages is stopped, and the counts are reset. The same drive operation is performed for the second and following lines, whereby first imaging signals (R signals, G signals, and B signals) corresponding to the amounts of charges stored in the floating gates FG1′ of all the lines are output.

Then, the reading control sections 20a′ turn on the transistors 20f′ to precharge the column signal lines 12′. Then, the reading control sections 20a′ turn on the transistors 20e′ to establish electrical continuity between the column signal lines 12′ and the sense amplifiers 20b′, respectively. In this state, the ramp-up circuits 20d′ start to apply ramp voltages (Vth reading voltages) to the control gates CG2′ of the pixel portions 100′ of the first line (after the start of application of the ramp voltages, the counts are incremented from an initial value (e.g., “0”)). Counts corresponding to values of ramp voltages that are applied when the drain potentials of the nonvolatile memory transistors MR2′ of the pixel portions 100′ of the first line drop are latched in the reading circuits 20′, respectively, and the latched counts are output from the output amplifier 60′ via the signal line 70′ under the control of the horizontal shift register 50′ as imaging signals that were generated by the photoelectric conversion portions 3′ during the exposure period when the special light-1, 2, and 3 were emitted and were stored there. After the output of the imaging signals, the transistors 20f′ are turned off, the application of the ramp voltages is stopped, and the counts are reset. The same drive operation is performed for the second and following lines, whereby second imaging signals (special light-1 signals, special light-2 signals, and special light-3 signals) corresponding to the amounts of charges stored in the floating gates FG2′ of all the lines are output.

After the output of the second imaging signals, the control section 40′ sets the potentials of the control gates CG1′ and CG2′ of all the pixel portions 100′ at −Vcc and sets the potential of the semiconductor substrate K′ at Vcc. As a result, the charges stored in the floating gates FG1′ and FG2′ are drawn into the semiconductor substrate K′ and thereby erased.

The above operation is performed in one frame period. As described above, the number of transistors can be decreased by using the nonvolatile memory transistors MT1′ and MT2′ as the plural charge storage portions provided in each pixel portion 100′.

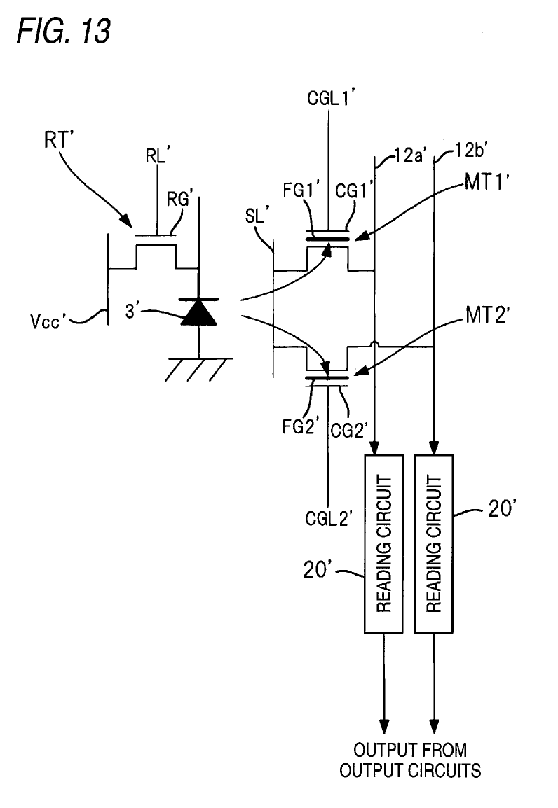

In the example of FIGS. 8A and 8B, the nonvolatile memory transistors MT1′ and MT2′ are connected to the single, common column signal line 12′, which is connected to the single reading circuit 20′. Alternatively, as shown in FIG. 13, a configuration is possible in which the nonvolatile memory transistors MT1′ and MT2′ are connected to different column signal lines 12a′ and 12b′ and the column signal lines 12a′ and 12b′ are connected to respective reading circuits 20′. Output circuits are provided for the two respective sets of reading circuits 20′, whereby first imaging signals and second imaging signals can be read out parallel from the solid-state imaging device. This makes it possible to shorten the time that is taken from shooting to image display or recording.

The solid-state imaging device 10 of FIG. 2A in which each charge storage portion is formed by two transistors may also be modified so that the read transistors RT1 and RT2 are connected to different signal lines and those signal lines are connected to respective reading circuits 20.

The solid-state imaging device 10 of FIG. 2A in which each charge storage portion is formed by two transistors may also be modified so that the area other than the photoelectric conversion portion 11 is shielded from light by a light shield layer and photoelectric conversion portion 11 is formed so as to extend to under the channel regions of the write transistors WT1 and WT2. This makes it possible to increase the charge injection efficiency.

Modifications of the endoscope apparatus of FIG. 1 will be described below.

### (First Modification)

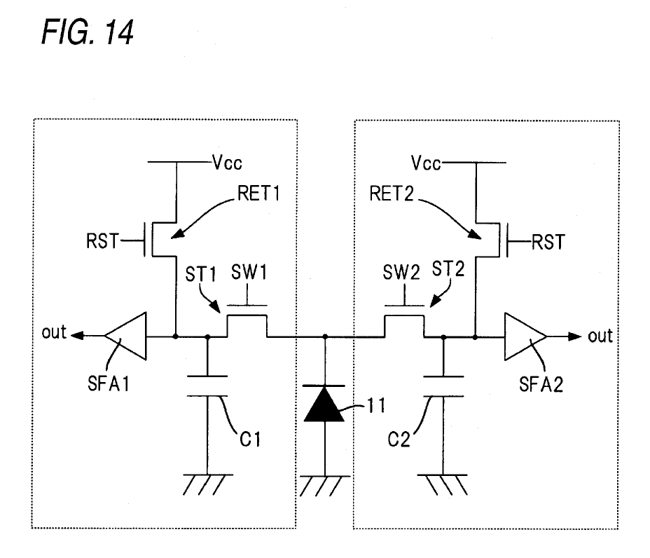

FIG. 14 shows a modification of the endoscope apparatus of FIG. 1 and is an equivalent circuit diagram showing a modified structure of each pixel portion of the solid-state imaging device 10 of FIG. 2A.

As shown in FIG. 14, each pixel portion is provided with floating diffusion capacitors C1 and C2 as plural charge storage portions a selected one of which can store charge generated by the photoelectric conversion portion 11. As shown in FIG. 14, each pixel portion is also provided with a switch transistor ST1, a reset transistor RET1, and a source follower amplifier SFA1 which correspond to the floating diffusion capacitor C1 and a switch transistor ST2, a reset transistor RET2, and a source follower amplifier SFA2 which correspond to the floating diffusion capacitor C2.

The switch transistor ST1 controls transfer of charge from the photoelectric conversion portion 11 to the floating diffusion capacitor C1. The source follower amplifier SFA1 is connected to the floating diffusion capacitor C1 and outputs a signal corresponding to the amount of charge transferred to the floating diffusion capacitor C1. The reset transistor RET1 resets the potential of the floating diffusion capacitor C1 to a power source voltage Vcc.

The switch transistor ST2 controls transfer of charge from the photoelectric conversion portion 11 to the floating diffusion capacitor C2. The source follower amplifier SFA2 is connected to the floating diffusion capacitor C2 and outputs a signal corresponding to the amount of charge transferred to the floating diffusion capacitor C2. The reset transistor RET2 resets the potential of the floating diffusion capacitor C2 to the power source voltage Vcc.

In the endoscope apparatus incorporating the solid-state imaging device having the pixel portions shown in FIG. 14, first, in response to a shooting instruction, the switch transistors ST1 and the reset transistors RET1 of all the pixel portions are turned on. As a result, the unnecessary charge stored in each photoelectric conversion portion 11 is transferred entirely to the floating diffusion capacitor C1 and then ejected to the drain of the reset transistor RET1. Then, the switch transistors ST1 and the reset transistors RET1 of all the pixel portions are turned off and, at the same time, an exposure using white light is started. Upon termination of an exposure period, the switch transistors ST1 of all the pixel portions are turned on and charge generated by each photoelectric conversion portion 11 is transferred entirely to the floating diffusion capacitor C1. Then, the switch transistors ST1 are turned off.

Subsequently, the switch transistors ST2 and the reset transistors RET2 of all the pixel portions are turned on. As a result, the residual charge remaining in each photoelectric conversion portion 11 is transferred entirely to the floating diffusion capacitor C2 and then ejected to the drain of the reset transistor RET2. Then, the switch transistors ST2 and the reset transistors RET2 of all the pixel portions are turned off and, at the same time, an exposure using special light beams is started. Upon termination of an exposure period, the switch transistors ST2 of all the pixel portions are turned on and charge generated by each photoelectric conversion portion 11 is transferred entirely to the floating diffusion capacitor C2. Then, the switch transistors ST2 are turned off.

Upon completion of the charge storage, a drive for reading, to the outside, using the source follower amplifiers SFA1, imaging signals corresponding to the amounts of charges transferred to the floating diffusion capacitors C1 is performed for all the lines, whereby imaging signals are read out. Then, a drive for reading, to the outside, using the source follower amplifiers SFA2, imaging signals corresponding to the amounts of charges transferred to the floating diffusion capacitors C2 is performed for all the lines, whereby imaging signals are read out.

The above configuration also makes it possible to shorten the interval between two shooting operations while suppressing heat generation and hence to miniaturize the apparatus and increase the diagnosis accuracy.

### (Second Modification)

In the solid-state imaging device 10 incorporated in the imaging device of FIG. 1, the color filters have a Bayer arrangement. When attention is paid to four (two-row/two-column) pixel portions 100, there are two pixel portions 100 having a green color filter (see FIG. 7). Therefore, no problems arise even if one of those two pixel portions is used as a pixel portion for detecting another wavelength component.

In the second modification, the green color filter of one of the two pixel portions 100 having a green color filter among each set of four (two-row/two-column) pixel portions of the solid-state imaging device 10 is changed to a filter which cuts light in the red, green, and blue wavelength ranges and transmits special light-4 whose emission wavelength is set so that the wavelength of reflection light or excitation-induced light coming from an object will be outside the red, green, and blue wavelength ranges. This configuration may be such that the emission wavelength of the special light-4 is located on the longer-wavelength side (e.g., 800 nm) of the red wavelength range (see FIG. 5) and that an infrared transmission filter is employed which transmits light in an infrared wavelength range (longer than or equal to 700 nm) and cuts light in the red, green, and blue wavelength ranges. Another configuration is possible in which the emission wavelength of the special light-4 is located on the shorter-wavelength side (e.g., 300 nm) of the blue wavelength range and an ultraviolet transmission filter is employed which transmits light in an ultraviolet wavelength range (shorter than or equal to 350 nm) and cuts light in the red, green, and blue wavelength ranges.

An LED for emitting special light-4 is added to the special light unit of the light source 1.

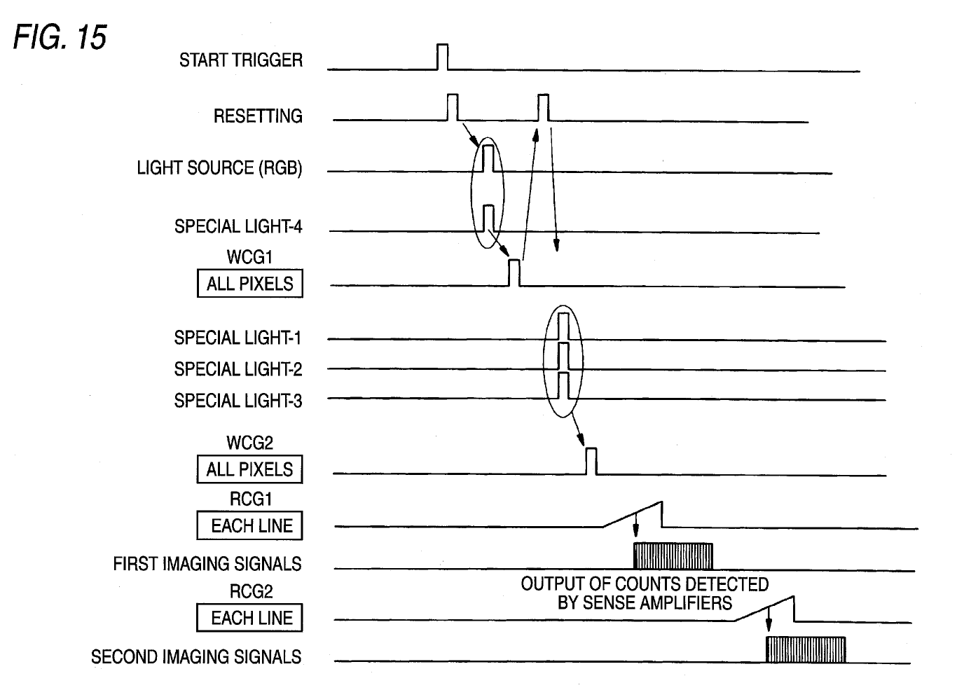

The operation of the thus-configured endoscope apparatus will be described below. FIG. 15 is a timing chart showing an operation of the second modification of the imaging apparatus of FIG. 1.

When an instruction to shoot an object is issued by manipulation of the manipulation unit 25, the instruction is input to the system control section 24 and the system control section 24 gives a shooting instruction to the solid-state imaging device 10.

When the solid-state imaging device 10 receives the shooting instruction, triggered by the shooting instruction the control section 40 supplies reset pulses to the reset gates RG of the reset transistors RET of all the pixel portions 100. As a result, the unnecessary charges stored in the photoelectric conversion portions 11 are ejected to the drains of the reset transistors RET.

Upon completion of the resetting, the system control section 24 issues an instruction to the light source drive section 21 and thereby causes the halogen lamp 1a and the LED for emitting special light-4 to emit white light and special light-4, respectively. As a result, light including light components in the red, green, and blue wavelength ranges and special light-4 is emitted from the light source 1. Although in FIG. 9 light (white light and special light-4) is emitted with a short delay from the supply of the reset pulses, it is preferable that this light emission be performed simultaneously with the completion of the resetting.

This light emission is performed for, for example, an exposure time that is set in the endoscope apparatus. During the white light emission period, in each pixel portion 100 having a red color filter of the solid-state imaging device 10, only red light of light coming from an object passes through the red color filter and enters the photoelectric conversion portion 11, where charge corresponding to the amount of the red light is generated and stored. In each pixel portion 100 having a green color filter of the solid-state imaging device 10, only green light of light coming from the object passes through the green color filter and enters the photoelectric conversion portion 11, where charge corresponding to the amount of the green light is generated and stored. In each pixel portion 100 having a blue color filter of the solid-state imaging device 10, only blue light of light coming from the object passes through the blue color filter and enters the photoelectric conversion portion 11, where charge corresponding to the amount of the blue light is generated and stored. In each pixel portion 100 having an infrared transmission filter of the solid-state imaging device 10, only reflection light or excitation-induced light corresponding to the special light-4 of light coming from the object passes through the infrared transmission filter and enters the photoelectric conversion portion 11, where charge corresponding to the amount of the reflection light or excitation-induced light corresponding to the special light-4 is generated and stored.

Upon termination of the exposure period, the control section 40 supplies write pulses to the write control gates WCG1 of all the pixel portions 100 and thereby causes the floating gates FG1 to store the charges that were generated by the photoelectric conversion portions 11 during the exposure period. The supply of write pulses may be started either simultaneously with the end of the exposure period or simultaneously with the start of the exposure period (finished simultaneously with the end of the exposure period).

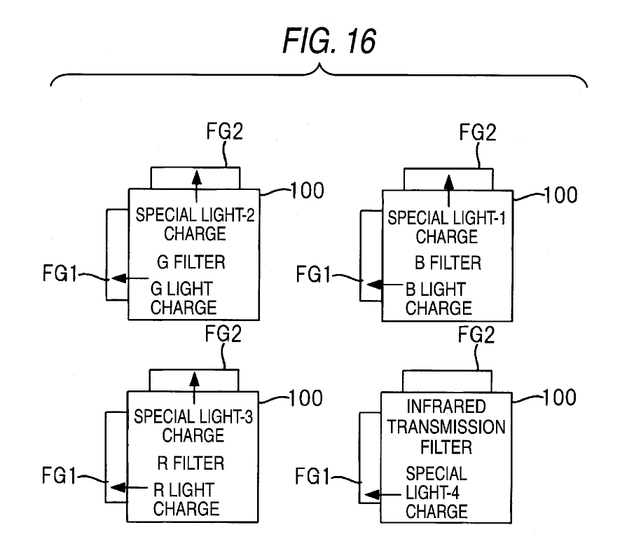

As shown in FIG. 16, upon the supply of the write pulses, the charges generated in the respective pixel portions 100 (i.e., the charges generated by the red light, the charges generated by the green light, the charges generated by the blue light, and the charges generated by the special light-4) are stored in the floating gates FG1 of the respective pixel portions 100.

Upon completion of the charge storage in the floating gates FG1, the control section 40 again supplies reset pulses to the reset gates RG of the reset transistors RET of all the pixel portions 100. As a result, the residual charges remaining in the photoelectric conversion portions 11 without being injected into the floating gates FG1 are ejected to the drains of the reset transistors RET.

After completion of the second resetting, the system control section 24 issues an instruction to the light source drive section 21 to cause the special light unit to emit special light beams (special light-1, 2, and 3). Although in FIG. 15 special light beams are emitted after a short delay from the supply of the reset pulses, it is preferable that special light beams be emitted simultaneously with the completion of the resetting.

The special light emission is performed for, for example, an exposure time that is set in the endoscope apparatus. During the special light emission period, in each pixel portion 100 having a red color filter of the solid-state imaging device 10, only reflection light or excitation-induced light corresponding to the special light-3 of light coming from an object passes through the red color filter and enters the photoelectric conversion portion 11, where charge is generated and stored. In each pixel portion 100 having a green color filter of the solid-state imaging device 10, only reflection light or excitation-induced light corresponding to the special light-2 of light coming from the object passes through the green color filter and enters the photoelectric conversion portion 11, where charge is generated and stored. In each pixel portion 100 having a blue color filter of the solid-state imaging device 10, only reflection light or excitation-induced light corresponding to the special light-1 of light coming from the object passes through the blue color filter and enters the photoelectric conversion portion 11, where charge is generated and stored.

Upon termination of the exposure period, the control section 40 supplies write pulses to the write control gates WCG2 of all the pixel portions 100 and thereby causes the floating gates FG2 to store the charges that were generated by the photoelectric conversion portions 11 during the exposure period. The supply of write pulses may be started either simultaneously with the end of the exposure period or simultaneously with the start of the exposure period (finished simultaneously with the end of the exposure period).

As shown in FIG. 16, upon the supply of the write pulses, the charges generated in the respective pixel portions 100 (i.e., the charges generated by the special light-1, the charges generated by the special light-2, and the charges generated by the special light-3) are stored in the floating gates FG2 of the respective pixel portions 100.

After completion of the charge storage in the floating gates FG2, the control section 40 sets the drain potentials of the read transistors RT1 of the pixel portions 100 of the first line at Vr (<Vcc) and starts to apply ramp voltages to the read control gates RCG1 of the pixel portions 100 of the first line. Counts corresponding to values of ramp voltages that are applied when the drain potentials of the read transistors TR1 of the first line drop are latched in the reading circuits 20, respectively, and the latched counts are output from the output amplifier 60 as imaging signals. The control section 40 performs the same drive operation for the second and following lines and thereby causes output of first imaging signals (R signals, G signals, B signals, and special light-4 signals) corresponding to the amounts of charges stored in the floating gates FG1 of all the lines.

Then, the control section 40 sets the drain potentials of the read transistors RT2 of the pixel portions 100 of the first line at Vr (<Vcc) and starts to apply ramp voltages to the read control gates RCG2 of the pixel portions 100 of the first line. Counts corresponding to values of ramp voltages that are applied when the drain potentials of the read transistors TR2 of the first line drop are latched in the reading circuits 20, respectively, and the latched counts are output from the output amplifier 60 as imaging signals. The control section 40 performs the same drive operation for the second and following lines and thereby causes output of second imaging signals (special light-1 signals, special light-2 signals, and special light-3 signals) corresponding to the amounts of charges stored in the floating gates FG2 of all the lines.

After the output of the second imaging signals, the control section 40 sets the potentials of the write control gates WCG1 and WCG2 and the read control gates RCG1 and RCG2 of all the pixel portions 100 at −Vcc and sets the potential of the semiconductor substrate at Vcc. As a result, the charges stored in the floating gates FG1 and FG2 are drawn into the semiconductor substrate and thereby erased.

The above operation is performed in one frame period.

As described above, the endoscope apparatus according to the second modification can produce RGB color image data using white light and also produce image data using special light-1, 2, 3, and 4 (five kinds of image data in total). The endoscope apparatus can thereby increase the efficiency of a diagnosis made by a doctor.

In the above description, the number of kinds of image data is increased by one by forming pixel portions having a filter that transmits special light-4. However, a configuration in which such pixel portions have no filter is defined as one version of the configuration in which filters that transmit special light-4 are provided. In the configuration in which the pixel portions corresponding to the special light-4 are not provided with any filter, in the example operation of FIG. 15, charges corresponding to the amounts of received white light and special light-4 are stored in the floating gates FG1 of the pixel portions having no filter and charges corresponding to the amounts of special light-1, charges corresponding to the amounts of special light-2, and charges corresponding to the amounts of special light-3 are stored in the floating gates FG2. Therefore, only shot image data corresponding to the special light-4 can be extracted by removing a white light component from an imaging signal that is read out from the floating gate FG1 of each pixel portion having no filter using R, G, and B signal components of the adjacent pixel portions. Furthermore, only shot image data corresponding to the special light-1 can be extracted by removing a special light-2 component and a special light-3 component of the adjacent pixel portions from an imaging signal that is read out from the floating gate FG2 of each pixel portion having no filter. In this configuration, whereas calculation is necessary for extracting imaging signals corresponding to special light-4, no calculation is necessary for imaging signals corresponding to white light and imaging signals corresponding to special light-1, imaging signals corresponding to special light-2, and imaging signals corresponding to special light-3. Therefore, the amount of calculation can be made much smaller than in a case that calculation is necessary for such imaging signals.

As described above, this specification discloses the following items:

A solid-state imaging device includes plural pixel portions and a signal reading section. Each of the plural pixel portions includes a photoelectric conversion portion, and plural charge storage portions a selected one of which can store charge generated in the photoelectric conversion portion. The signal reading section independently reads out signals corresponding to amounts of charges stored in the plural respective charge storage portions.

With this configuration, plural kinds of charges generated by plural times of exposures can be stored in each pixel portion in a distinguished manner. Therefore, it is not necessary to read out signals corresponding to generated charges every time an exposure is performed. That is, signals can be read out together after plural times of exposures. As a result, the intervals between plural times of exposures can be shortened and shooting operations using plural light sources can be performed successively in a short time. This makes it possible to take images of approximately the same object under different sets of conditions. This makes it possible to increase the diagnosis accuracy of an endoscope examination.

The solid-state imaging device may further include a charge storage control unit that independently performs controls for storing charges in the plural respective charge storage portions.

With this configuration, it is possible to store plural kinds of charges generated by plural times of exposures in each pixel portion in a distinguished manner.

In the solid-state imaging device, each pixel portion may further include charge ejecting unit that ejects charge stored in the photoelectric conversion portion.

In this configuration, even when exposures are performed plural times successively, unnecessary charges stored in the photoelectric conversion portions can be ejected before each exposure. As a result, part of charges generated by a preceding exposure are mixed with charges generated by the next exposure. Image quality degradation can thus be avoided.

The solid-state imaging device may be such that each of the plural charge storage portions is a transistor including a charge storage region formed above a semiconductor substrate in which the photoelectric conversion portion is formed, the charge is stored in the charge storage region, and the signal reading section is a signal reading circuit which reads out, as the signal, a change of a threshold voltage of the transistor, the change corresponding to an amount of charge stored in the charge storage region.

The solid-state imaging device may further include: a light shield layer that is formed above the semiconductor substrate, and has an opening above part of the photoelectric conversion portion. The charge storage region and a channel region of the transistor are covered with the light shield layer. And the photoelectric conversion portion extends to under the channel region of the transistor.

In this configuration, a part of the photoelectric conversion portion exists under the channel region of the transistor. Therefore, charge generated by the photoelectric conversion portion according to the amount of light incident through the opening of the light shield layer can be injected efficiently into a charge storage portion from the part, coextending with the channel region, of the photoelectric conversion portion via the channel region.

The solid-state imaging device may be such that the charge storage region is a floating gate.

In this configuration, one charge is stored in the floating gate, the charge is not prone to be influenced by noise coming from the neighborhood. The SN ratio can thus be increased.

The solid-state imaging device may be such that the transistor includes a write transistor for injecting the charge into the floating gate and a read transistor whose threshold value, a change of which is to be read out as the signal, varies according to a potential variation of the floating gate, and that the write transistor has a two-terminal structure having a gate and a source which is connected to the photoelectric conversion portion.

With this configuration, it is unnecessary to provide each pixel portion with a space for formation of the drain of the write transistor and hence makes it possible to increase the degree of freedom of the layout and accommodate increase in the number of pixels and the miniaturization.

The solid-state imaging device may be such that the plural transistors of each pixel portion are connected to different output signal lines to which respective signal reading circuits are connected.

With this configuration, it is possible to read out signals from the plural transistors in parallel and hence to increase the speed of a shooting operation.

The solid-state imaging device may be such that the plural charge storage portions are plural floating diffusion capacitors; that the charge storage control unit is transistors which are provided for the plural respective floating diffusion capacitors and transfer charges to the associated floating diffusion capacitors from the photoelectric conversion portion; and that the signal reading section is source follower amplifiers provided for the plural respective floating diffusion capacitors and output signals corresponding to changes of potentials of the associated floating diffusion capacitors.

With this configuration, it is possible to store plural kinds of charges generated by plural times of exposures in each pixel portion in a distinguished manner.

It is also disclosed that an endoscope apparatus including any of the above solid-state imaging devices; a light source capable of emitting plural kinds of light; and drive unit for performing drives for causing emissions of the plural kinds of light at different time points in response to a shooting trigger, causing charges generated in the photoelectric conversion portion by incident light beams coming from an object as a result of the emissions to be stored in the different charge storage portions corresponding to the plural respective kinds of light in synchronism with the emissions of the plural kinds of light, and causing the signal reading section to start reading out signals corresponding to amounts of the charges stored in the plural charge storage portions, respectively.

In this configuration, since signals are read out together after plural times of exposures using plural kinds of light, shooting operations using plural kinds of light can be performed successively in a short time. Therefore, shooting operations using plural kinds of light can be performed almost simultaneously and images of approximately the same object can be taken under different sets of conditions.

The endoscope apparatus may be such that the plural pixel portions include first pixel portions, second pixel portions, and third pixel portions; that each of the first pixel portions comprises a first color filter which is provided over the photoelectric conversion portion and transmits light in a red wavelength range; that each of the second pixel portions comprises a second color filter which is provided over the photoelectric conversion portion and transmits light in a green wavelength range; that each of the third pixel portions comprises a third color filter which is provided over the photoelectric conversion portion and transmits light in a blue wavelength range; that the plural kinds of light are light having components in the red, green, and blue wavelength ranges and special light; and that an emission wavelength of the special light is set so that reflection light or excitation-induced light coming from the object as a result of the emission of the special light will include at least one of a particular wavelength in the red wavelength range, a particular wavelength in the green wavelength range, and a particular wavelength in the blue wavelength range.

With this configuration, it is possible to perform, almost simultaneously, shooting using white light and shooting using special light without providing a spectral filter for special light.

The endoscope apparatus may be such that the plural pixel portions further include fourth pixel portions; that the plural kinds of light are light including first light having components in the red, green, and blue wavelength ranges and second light whose emission wavelength is set so that a wavelength of reflection light or excitation-induced light coming from the object will be outside the red, green, and blue wavelength ranges, and the special light; and that each of the fourth pixel portions comprises a filter which is provided over the photoelectric conversion portion and transmits the second light.

The endoscope apparatus may be such that the emission wavelength of the second light is set on the longer-wavelength side of the red wavelength range or on the shorter-wavelength side of the blue wavelength side.

It is also disclosed a drive method of a solid-state imaging device having plural pixel portions. Each of the pixel portions includes a photoelectric conversion portion and plural charge storage portions capable of storing, at different time points, charges generated in the photoelectric conversion portion. The drive method includes: causing emissions of plural kinds of light at different time points in response to a shooting trigger, and causing charges generated in the photoelectric conversion portion by incident light beams coming from an object as a result of the emissions to be stored in the different charge storage portions corresponding to the plural respective kinds of light in synchronism with the emissions of the plural kinds of light; and starting to read out signals corresponding to amounts of the charges stored in the plural charge storage portions, respectively.

What is claimed is:

1. A solid-state imaging device comprising:
plural pixel portions each comprising:
a photoelectric conversion portion; and
plural charge storage portions a selected one of which can store charge generated in the photoelectric conversion portion; and
a signal reading section that independently reads out signals corresponding to amounts of charges stored in the plural respective charge storage portions.

2. The solid-state imaging device according to claim 1, further comprising:
a charge storage control unit that independently performs controls for storing charges in the plural respective charge storage portions.

3. The solid-state imaging device according to claim 2, wherein each pixel portion further comprises charge ejecting unit that ejects charge stored in the photoelectric conversion portion.

4. The solid-state imaging device according to claim 1,
wherein: each of the plural charge storage portions is a transistor including a charge storage region formed above a semiconductor substrate in which the photoelectric conversion portion is formed;
the charge is stored in the charge storage region; and
the signal reading section is a signal reading circuit which reads out, as the signal, a change of a threshold voltage of the transistor, the change corresponding to an amount of charge stored in the charge storage region.

5. The solid-state imaging device according to claim 4, further comprising:
a light shield layer that is formed above the semiconductor substrate, and has an opening above part of the photoelectric conversion portion,
wherein the charge storage region and a channel region of the transistor are covered with the light shield layer, and
the photoelectric conversion portion extends to under the channel region of the transistor.

6. The solid-state imaging device according to claim 4,
wherein the charge storage region is a floating gate.

7. The solid-state imaging device according to claim 6,
wherein the transistor includes:
a write transistor that injects the charge into the floating gate; and
a read transistor whose threshold value, a change of which is to be read out as the signal, varies according to a potential variation of the floating gate, and
wherein the write transistor has a two-terminal structure having a gate and a source which is connected to the photoelectric conversion portion.

8. The solid-state imaging device according to claim 4,
wherein the plural transistors of each pixel portion are connected to different output signal lines to which respective signal reading circuits are connected.

9. The solid-state imaging device according to claim 2,
wherein the plural charge storage portions are plural floating diffusion capacitors;
the charge storage control unit is transistors which are provided for the plural respective floating diffusion capacitors and transfer charges to the associated floating diffusion capacitors from the photoelectric conversion portion; and
the signal reading section is source follower amplifiers that are provided for the plural respective floating diffusion capacitors and output signals corresponding to changes of potentials of the associated floating diffusion capacitors.

10. An endoscope apparatus comprising:
the solid-state imaging device according to claim 1;
a light source capable of emitting plural kinds of light; and
a drive unit that performs drives for
causing emissions of the plural kinds of light at different time points in response to a shooting trigger,
causing charges generated in the photoelectric conversion portion by incident light beams coming from an object as a result of the emissions to be stored in the different charge storage portions corresponding to the plural respective kinds of light in synchronism with the emissions of the plural kinds of light, and
causing the signal reading section to start reading out signals corresponding to amounts of the charges stored in the plural charge storage portions, respectively.

11. The endoscope apparatus according to claim 10,
wherein the plural pixel portions include first pixel portions, second pixel portions, and third pixel portions;
each of the first pixel portions comprises a first color filter which is provided above the photoelectric conversion portion and transmits light in a red wavelength range;
each of the second pixel portions comprises a second color filter which is provided above the photoelectric conversion portion and transmits light in a green wavelength range;
each of the third pixel portions comprises a third color filter which is provided above the photoelectric conversion portion and transmits light in a blue wavelength range;
the plural kinds of light are light having components in the red, green, and blue wavelength ranges and special light; and
an emission wavelength of the special light is set so that reflection light or excitation-induced light coming from the object as a result of the emission of the special light will include at least one of a particular wavelength in the red wavelength range, a particular wavelength in the green wavelength range, and a particular wavelength in the blue wavelength range.

12. The endoscope apparatus according to claim 11,
wherein: the plural pixel portions further include fourth pixel portions;
the plural kinds of light are light including first light having components in the red, green, and blue wavelength ranges and second light whose emission wavelength is set so that a wavelength of reflection light or excitation-induced light coming from the object will be outside the red, green, and blue wavelength ranges, and the special light; and
each of the fourth pixel portions comprises a filter which is provided above the photoelectric conversion portion and transmits the second light.

13. The endoscope apparatus according to claim 12,
wherein the emission wavelength of the second light is set on the longer-wavelength side of the red wavelength range or on the shorter-wavelength side of the blue wavelength side.

14. A drive method of a solid-state imaging device having plural pixel portions, wherein each of the pixel portions comprises a photoelectric conversion portion and plural charge storage portions capable of storing, at different time points, charges generated in the photoelectric conversion portion, the drive method comprising:
causing emissions of plural kinds of light at different time points in response to a shooting trigger, and causing charges generated in the photoelectric conversion portion by incident light beams coming from an object as a result of the emissions to be stored in the different charge storage portions corresponding to the plural respective kinds of light in synchronism with the emissions of the plural kinds of light; and
starting to read out signals corresponding to amounts of the charges stored in the plural charge storage portions, respectively.
